(19) United States
(12) Patent Application Publication
(10) Pub. No.: US 2009/0068656 A1
(43) Pub. Date: Mar. 12, 2009

(54) Methods of diagnosing osteoarthritis

(76) Inventors: Frank Beier, London (CA); Thomas Appleton, Ailsa Craig (CA); James Henry, Burlington (CA)

Correspondence Address: VALENTINE A COTTRILL;SUSAN TANDAN, 50 QUEEN STREET NORTH, STE. 1020, P.O. BOX 2248, KITCHENER, ON, N2H6M2, CA

(21) Appl. No.: 11/905,442

(22) Filed: Oct. 1, 2007

Related U.S. Application Data
(60) Provisional application No. 60/848,365, filed on Oct. 2, 2006.

(51) Int. Cl. C12Q 1/68; C12Q 1/02

(52) U.S. Cl. 435/6; 435/29

(57) ABSTRACT

A method of diagnosing osteoarthritis in a mammal is provided comprising the steps of obtaining a biological sample from the mammal; and quantifying in the sample the expression of at least one chondrocyte-specific gene or gene product, wherein a differential in expression of said gene or gene product in comparison with expression of said gene or gene product in a non-osteoarthritic mammal is indicative of osteoarthritis.

### FIELD OF THE INVENTION

The present invention relates to osteoarthritis and methods of diagnosing osteoarthritis. In particular, the invention relates to methods in which chondrocyte-specific genes and gene products may be utilized to diagnose osteoarthritis.

### BACKGROUND OF THE INVENTION

Osteoarthritis (OA) is the most common degenerative joint disease in the world. Although many environmental and behavioural factors have been correlated with the onset of OA, its etiology is largely unknown. Understanding of this disease is confounded by the fact that OA appears to be affected differently by various influences in each case. Secondary OA arises from an initial trauma (such as a ligament tear) resulting in joint instability, unnatural articulation and eventual development of pathology (1, 2). Secondary OA represents a major proportion of OA cases and commonly affects people in their 30s and 40s (3). Less obvious obtrusive causes, including genetic factors, result in idiopathic or primary OA) (4, 5).

One characteristic of OA is articular cartilage degradation. It is well established that the cells of hyaline cartilage (chondrocytes) produce and maintain their surrounding extracellular matrix (6, 7). The homeostasis of cartilage matrix metabolism relies on the catabolism of matrix proteins such as type II collagen and aggrecan and subsequent replacement of digested proteins with new protein synthesized by chondrocytes (8, 9). Catabolic events are largely due to proteolytic enzymes of the matrix metalloproteinase (MMP) and aggrecanase families (10, 11). Overall, a balance between synthesis and degradation is established, maintaining a healthy cartilage. During OA pathogenesis, however, the balance shifts towards degradation.

Cartilage homeostasis is tightly regulated through intercellular signaling between chondrocytes. Chondrocytes produce and respond to signaling molecules including cytokines and growth factors to direct cell metabolism. For example, TGF-β (transforming growth factor-beta) signaling in chondrocytes promotes type II collagen production and inhibits collagen cleavage (12, 13). Conversely, cytokines such as TNF-α (tumour necrosis factor-alpha) and IL-1β (interleukin-1 beta) promote the production of proteases that degrade cartilage (14, 15). However, these examples represent only a small portion of the complex interactions responsible for regulating chondrocyte function.

Thus, it is desirable to understand to a greater extent the complement of factors that are disrupted in OA. Genome-wide analyses of dysregulated genes in OA constitute a step in this direction. Of particular interest is the elucidation of molecular changes during the onset of OA, in order to develop strategies that allow early detection and intervention.

### SUMMARY OF THE INVENTION

The expression pattern of multiple chondrocyte-specific genes and gene products has now been determined which is useful in the diagnosis of osteoarthritis.

Thus, in one aspect of the invention, thus, there is provided a method of diagnosing osteoarthritis in a mammal comprising the steps of:

i) obtaining a biological sample from the mammal; and

ii) quantifying the expression of at least one chondrocyte-specific gene or gene product in the sample, wherein differential expression of said gene or gene product in comparison with a standard is indicative of osteoarthritis.

In another aspect of the invention, a kit for diagnosing osteoarthritis in a mammal is provided. The kit comprises at least one probe directed to a chondrocyte-specific gene in the mammal that exhibits differential expression of at least about 1.5-fold in comparison with a standard.

In another aspect of the invention, a method of diagnosing osteoarthritis in a mammal comprising:
i) obtaining a biological sample from the mammal; and
ii) quantifying in the sample the expression of multiple chondrocyte-specific genes or gene product products to generate a chondrocyte-specific gene or gene product expression profile; and
iii) comparing the generated profile with a standard profile, wherein differential expression of one or more of said genes or gene products is indicative of osteoarthritis.

These and other aspects of the invention will become apparent from the description that follows, as well as the figures described below.

### DESCRIPTION OF THE FIGURES

FIG. 1 illustrates microarray analyses of cartilage gene expression in a rat model of OA including a dendogram illustrating the results of a clustering analysis to distinguish the expression profiles of ipsilateral, contralateral and sham expression (A); results of contralateral and ipsilateral expression of known marker genes of OA in the osteoarthritic model (B) and similar analyses for selected extracellular matrix (ECM) gene expression in contralateral and ipsilateral OA cartilage samples (C);

FIG. 2 graphically illustrates real-time PCR validation of microarray expression profiles;

FIG. 3 illustrates the real-time PCR analysis of cathepsin C (Ctsc) (A) and chemokine receptor 4 (Cxcr4) (B) gene expression profiles; and

FIG. 4 is an analysis of microarray results including: a comparison of differential articular chondrocyte gene expression in ipsilateral OA and contralateral cartilage (A); a comparison of the identity of differentially expressed genes between ipsilateral OA and contralateral cartilage (B); a gene ontology analysis of differentially expressed genes in ipsilateral OA cartilage (C); and a comparison of the distribution of genes involved in specific categories with relevance in OA (D).

### DETAILED DESCRIPTION OF THE INVENTION

A method of diagnosing osteoarthritis in a mammal is provided. The method comprises the steps of obtaining a biological sample from the mammal and quantifying the expression of at least one chondrocyte-specific gene or gene product in the sample. The detection of differential expression of the chondrocyte-specific gene or gene product in comparison with a standard is indicative of osteoarthritis.

The term “biological sample” as it is used herein is meant to refer to any sample that may be obtained from a mammal to be diagnosed containing either targeted chondrocyte-specific genes or gene products. Preferably, the biological sample is obtainable non-invasively. Accordingly, although cartilage samples may be obtained and used in the present diagnostic methods, preferred biological samples include fluid samples such as blood, urine and other fluids that may contain the targetted gene or gene product. As one of skill in the art will appreciate, the appropriate biological sample may vary with the gene or gene product to be measured.

The term “chondrocyte-specific gene” refers to a gene expressed in the cartilage associated with a joint. Differential expression of genes normally expressed in the cartilage has been determined to be linked to osteoarthritis. Examples of chondrocyte-specific genes include genes encoding cytokines such as Bmp3, Bmp4, Cel2, Cklf1, Cklf1, Ddt, F3, Sppl and Tnfsf11; genes encoding growth factors such as Dtr, Egf, Esm1, Hdgfrp3, Igf1, Igf2, Igf2r, Igfbp6, Inhba, Ltbp1, Ltbp2, Nudt6, Pdgfrb, Tgfa, Tgfb2 and Wisp2; genes encoding insulin binding proteins such as Irs3; genes encoding notch binding proteins such as Jagged 1 and genes encoding proteins such as Kit ligands. Other chondrocyte-specific genes in accordance with the present methods include those encoding the following proteins: Tgfa, Ednra, Ctsc, Cxcr4, Inhba, Sfrp4 and Ccl2, as well as Idb4, Crabp2, Cd44, Rgs4, Rgs5, Tnfrsf12a, Klf15, Ramp2, Ramp3, Per2, Per3, Nr1d1, Nr1d2, Edg1, and I12rg. Also included are genes encoding Agtr1a, Aldh1a3, Aqp1, Aqp3, Arf6, Arl4, Arnt1, Basp1, Bmp3, Bst1, Casp12, Casq2, Cd1d1, Cdh2, Cdh11, Cdh13, Cd53, Cd74, Cklf1, Ctsh, Ctss, Cybb, Daf, Etl, Dtr, Gadd45a, Gap43, Gas7, Gucy1a3, Gucy1b3, Kdr, Mmp12, Nbl1, Nfil3, Pde8a, Panx3, Pdgfrb, Pdlim1, Phex, Prrx2, Rab38, Serpina1, Serpinf1, Serping1, Sfrp4, Stk17b, Tfpi2, Tnfip6, Tnfsf11, Wisp2, Ace, Agt, Agtr2, Dlx5, Egf, Egr1, Fgfr2, Gas6, Ghr, Gpr3711, Gstm1, Gstm³, Gstm5, Gstp2, Has2, Hnf3b, Hs3st1, I11r2, Irs3, Map2k6, Nfia, Nfib, Nr1d1, Nr1d2, Nr3c2, Nr4a1, Pias3, Pim3, Penk-rs, Pou3B, Ptgds, Ptgis, Ptprr, Rb12 and Rbp4. The expanded form of each acronym is set out in the appended Tables.

Preferred chondrocyte-specific genes for the purposes of diagnosing osteoarthritis include genes that encode extra-cellular matrix proteins that are secreted on expression, or genes that encode cell surface proteins, such as receptors. These proteins are preferred as diagnostic markers since they may be more readily identified in non-invasively obtained biological samples such as serum or urine samples. Examples of such preferred genes include genes encoding Cklf1, Inhba, Ccl2, Sfrp4, Ctsh, Ctss, Il2rg, Cd44, Pdgfrb, Fgfr2, Wisp2 and Tgfα.

Additional gene products that have been found to exhibit increased expression in osteoarthritis and are therefore indicative of osteoarthritis include chemokine signaling factors such as chemokine-like factor 1 (Ckl1), chemokine (C—X3-C) ligand 1 (Cxc3) and chemokine (C—X—C motif) receptor 4 (Cxcr4), as well as endothelin receptor type A (ednra). Examples of other chondrocyte specific genes and of chondrocyte specific gene products in accordance with the present invention are found in Table 1, Table 2 and Table 3.

The term “differential expression” as used herein with respect to genes and gene products refers to expression of a given gene or gene product in a mammal that differs from the expression of that gene/gene product in a healthy mammal. Thus, the level of expression of a given gene or gene product in a healthy non-osteoarthritic mammal is the standard against which the level of expression of the gene or gene product from a mammal to be diagnosed is compared. Differential expression may refer to down-regulation of a gene or inhibition of a gene product, or it may refer to up-regulation of a gene or over-expression of a gene product. The nature of the differential expression will vary with the specific gene/gene product. Although the magnitude of the differential expression will vary from gene/gene product to gene/gene product and is not particularly restricted, preferably the differential expression varies by at least about 1.5 fold from the standard magnitude of expression that generally occurs in a healthy non-osteoarthritic mammal.

As one of skill in the art will appreciate, the magnitude of differential expression of a chondrocyte-specific gene/gene product may vary at different stages of the disease state. For example, at early onset of osteoarthritis, for example 2 weeks, the expression of a given chondrocyte-specific gene may not indicate any differential expression in comparison to a standard; however, at 4 weeks following onset, the expression of the chondrocyte-specific gene may be differentially expressed and at 8 weeks following onset, this differential expression may be further differentially expressed. The present method relates to diagnosis of osteoarthritis at an early stage of the disease, for example, 2-8 weeks following onset.

Detection of chondrocyte-specific genes may be conducted using established techniques as is described in the specific examples that follow, including, for example nucleic acid probing techniques and techniques utilizing the polymerase chain reaction. Detection of chondrocyte-specific gene products is also conducted using established techniques as described including immunological techniques such as the use of primary antibodies to the gene product of interest.

Given the number of chondrocyte-specific genes that may be used to diagnose osteoarthritis, the invention also provides a method of diagnosing osteoarthritis in a mammal in which a profile of the expression of multiple chondrocyte-specific genes or gene products in a biological sample obtained from the mammal is generated and compared with a standard profile of chondrocyte-specific gene/gene product expression in a healthy non-osteoarthritic mammal. The profile will include the expression data of multiple chondrocyte-specific genes/gene products as identified above. Detection of differential expression of one or more chondrocyte-specific gene/gene products is indicative of osteoarthritis. An example of such profiling follows in the specific examples.

Kits for conducting the diagnostic methods of the present invention are also provided comprising a probe or probes directed to the chondrocyte-specific gene(s) targeted in the diagnostic method. Thus, a chondrocyte-containing biological sample obtained from a mammal is probed using the nucleic acid probe(s) of the kit to identify and/or quantify a chondrocyte-specific gene or gene profile that can be compared to a standard profile in order that a diagnosis can be made.

Diagnosis may also be made by identifying and quantifying one or more chondrocyte-specific gene products in a biological sample obtained from a mammal. The nature of the product to be quantified will dictate the identification and quantification techniques to be used in the determination. The results of the determination are then compared with standard values obtained from healthy non-osteoarthritic individuals. Deviation from these values, either higher or lower, is indicative of osteoarthritis.

Embodiments of the present invention are described by reference to the following specific examples which are not to be construed as limiting.

### EXAMPLE 1

### Materials & Methods

### Animal Model

Surgery was performed on the right knee of male Sprague Dawley rats of 300-325 g body weight (Charles River Laboratories, St. Constant, QU). Anaesthesia was induced with a ketamine/xylazine mix in saline (100 ul/100 g body weight), and Trisbrissen antibiotic (100 ul/100 g) was administered (Schering Canada, Inc, Pte. Claire, QU). The animals were randomly placed into two groups. The first group underwent anterior cruciate ligament transection (ACL-T) and partial medial meniscectomy (PMM) via an incision on the medial aspect of the right knee joint capsule, anterior to the medial collateral ligament. This treatment was used to induce OA pathogenesis (ipsilateral joint; the left knee joint is referred to as contralateral). The second group underwent a ‘sham’ operation during which a similar incision in the right joint capsule was made, but neither ACL-T nor PMM were performed. The animals underwent 30 minutes of forced joint mobilization on a rotarod apparatus 3 times per week for 28 days, at which point the study was terminated for cartilage harvest. This study was approved by the Animal Care and Use Committee at the University of Western Ontario.

### RNA Isolation & Preparation

Articular cartilage was dissected from each knee, cleaned of non-cartilaginous tissue and immersed in QIAzol (QIAgen, Mississauga, ON). It was necessary to pool cartilage from the femoral condyles and tibial plateaus to obtain enough RNA. After homogenization, total RNA was isolated using the Lipid Tissues Mini Kit (QIAgen) according to the manufacturer's protocol. RNA quantity was assessed using RiboGreen Assay (Molecular Probes, Burlington, ON), and RNA quality was confirmed using an Agilent 2100 Bioanalyzer (Agilent Technologies, Palo Alto, Calif.).

### Microarrays

Total RNA from the articular cartilage of 5 ipsilateral, 5 contralateral and 5 sham knee joints was hybridized to RAE230_{—}2.0 Affymetrix GeneChips® containing 31,099 probes. Each joint comprised one sample that was hybridized to a separate chip (n=5 per condition). Sample labeling, hybridization and detection were carried out according to the manufacturer's protocols (http://www.affymetrix.com/support/technical/manuals.affx) at the London Regional Genomics Center.

### Data Analyses

GeneSpring 7.2. Gene expression raw data files from Affymetrix GeneChips® were imported into GeneSpring 7.2 software (Silicon Genetics, Redwood City, Calif.). Raw data transformation set values less than 0.01 to 0.01, per-chip normalization was set to the 50^{th} percentile, and per-gene normalization was set to the median and specific samples. Data sets from the sham replicates were assigned to the ‘Normal’ treatment group and thus defined baseline expression for each probe. The remaining replicate data sets (contralateral and ipsilateral) were assigned to ‘Diseased’ treatment groups, averaged, and used in subsequent analysis. All data was interpreted using the log-ratio setting. From the starting list of 31,099 probes, 17,597 probes were determined to have a reliable signal using the GeneSpring 7.2 SG1a-1 signal intensity quality control script. The script was adjusted to require signal intensity above a threshold of 50 in at least 2 of the 3 conditions. The data was then passed through a parametric Welch's one-way ANOVA (analysis of variance) script, using a P value of ≦0.05 for statistical significance, reducing the list to 3,877 probes. Fold-change filtering was performed on this final list using GeneSpring 7.2 software.

Princival Component Analysis: GeneSpring 7.2 software was used to perform principal component analysis (PCA) on conditions using the list of 3,877 probe sets demonstrating significant signal in each sample as well as the 1.5-fold change filtered list of 1,619 probe sets.

Unsupervised Clustering: Raw data CEL files were imported into BRB Array Tools software (http://linus.nci.nih.gov.proxy1.lib.uwo.ca:2048/BRB-ArravTools.htm1) developed by Richard Simon and Amy Peng Lam. After log base 2 transformations, data were normalized by centering each array using the robust multi-chip average (RMA) algorithm. Intersection of the secondary lists resulted in 1,437 probe sets that demonstrated a consistently strong signal in each sample. These sets were used for agglomerative hierarchical clustering analysis in BRB Array Tools using centered metric correlation and average linkage.

Gene Ontology. After filtering probe sets using a minimum 1.5-fold change criterion for differential gene expression between sham and ipsilateral cartilage, the resulting list containing 1,619 probes was used in gene ontology (GO) analysis. Categorized lists were generated based on R. norvegicus annotations for biological process, cellular component, or molecular function GO's using FatiGO software (20, 21). The percentage of probes attributed to each category was then calculated relative to the total number of annotated probes used in the analysis.

### Real-time PCR Analysis

To quantitatively determine relative gene expression in cartilage RNA samples, real-time PCR was carried out as previously described (22). The reactions were prepared with the TaqMan® One-step Mastermix kit (Applied Biosystems). TaqMan® Gapdh (forward primer: 5′-GAAGGTGAAGGTCGGAGTC-3′ (SEQ ID NO: 1); reverse primer: 5′-GAAGATGGTGATGGGATTTC-3′ (SEQ ID NO: 2); probe: JOECAAGCTTCCCGTTCTCAGCC-TAMRA (SEQ ID NO: 3) control reagents were used as the internal control because they were less variable than 18S reagents. The target primer/probe sets for all tested genes were purchased as TaqMan® Gene Expression Assays (Applied Biosystems). An ABI Prism 7900 HT Real-Time PCR system (Perkin-Elmer) was used to detect amplification over 40 cycles for these experiments. Five independent RNA samples (from different animals than those used in the array experiments) were assayed from each treatment, each in quadruplicate. In each experiment, a negative control was used in the form of a reaction without template RNA. All relative expression values were calculated using the ACT method, normalized to Gapdh expression, and expressed in arbitrary units relative to the sham (control) expression values (set to 1). One way ANOVA was performed to determine statistical significance of the differences between means of each treatment type. A post-hoc Tukey's test was performed to compare the means of all treatment types. All expression values are displayed as the mean plus standard error of the mean (S.E.M.), and P<0.05 was considered statistically significant, indicated by a different letter (a, b, etc.). Analyses were carried out using GraphPad Prism 4 software (GraphPad Software, Inc., San Diego, Calif.).

### Immunofluorescence and Immunohistochemistry

Knee joints were obtained from rats 4 weeks post-surgery. Tissues were fixed via intracardial perfusion with 4% paraformaldehyde (PFA) and dissected. The joints were demineralized in 1% EDTA/glycerol for 4-5 weeks and embedded in paraffin. Sagittal sectioning of the decalcified knees was performed at the Robarts Research Institute Molecular Pathology Lab. Six micrometer sections from the medial compartment of each joint were used for immunohistochemical analyses. Primary antibody (Cedarlane Labs, Homby, ON) against MMP13 and secondary antibody conjugated to FITC were used to detect MMPI3 within articular cartilage of ipsilateral, contralateral, and sham knee joints. Toto-3 iodide (Molecular Probes, Burlington, ON) was used as a nuclear counterstain. Image development and confocal microscopy were performed using a Zeiss LSM510 META microscope and software (Carl Zeiss, Toronto, ON). These experiments were repeated 3 times with similar results; no signal was detected in samples without primary antibody. For immunohistochemistry, sections were probed with anti-cathepsin C or anti-CXCR4 primary antibodies (Abcam, Cambridge, Mass., USA) and secondary antibodies conjugated to HRP. Colourimetric detection with DAB substrate (Dako USA, Carpinteria, Calif.) was carried out for equal time periods for each section. Experiments for each protein were carried out on sections from at least 3 different animals with reproducible results.

### Results

### Microarray Analysis of Gene Expression in Articular Cartilage

Rats were analyzed four weeks post-surgery. At this time point, the ipsilateral OA knee joints show surface abrasions, edema of the superficial zone, and medial fissures when compared to sham and contralateral joints. Histological sections of sham, contralateral, and ipsilateral OA tibial plateaus 4 weeks post-surgery were stained with safranin-O (red-orange stain) for articular cartilage proteoglycans, fast green (green stain) for bone and fibrous tissue, and haematoxylin for nuclei. Cartilage degradation was indicated by surface discontinuity and proteoglycan depletion (loss of safranin-O stain) in ipsilateral samples, but not in the sham (control) or contralateral treatment.

RNA was isolated from the articular cartilage of sham (control), ipsilateral (OA) and contralateral knees and hybridized to Affymetrix RAE230_{—}2.0 GeneChips®. Data analysis with GeneSpring 7.2 resulted in a set of 3,877 probes expressed in at least two of the three treatment groups. To distinguish sham, contralateral and ipsilateral samples from one another, PCA was performed using GeneSpring 7.2 and 3 distinguishable clusters were identified. The ipsilateral cluster accounted for 62.83% of the variation among samples, while the contralateral and sham clusters accounted for 8.92% and 6.07% of the variation, respectively. These results indicate that all three samples have distinct gene expression profiles.

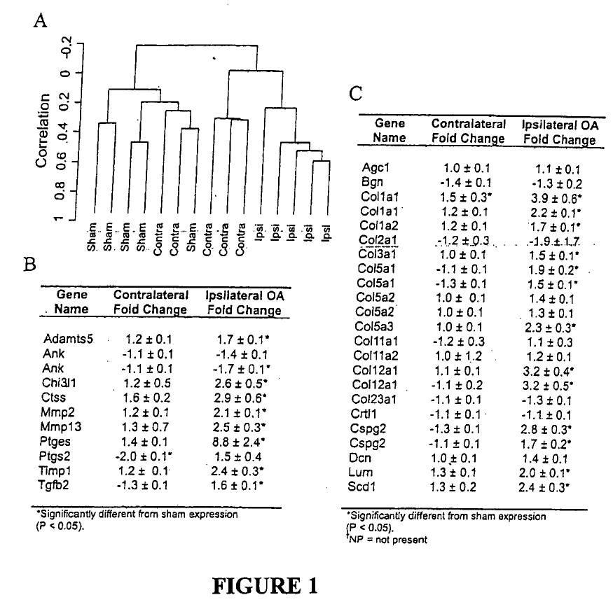

Unsupervised clustering was used to determine whether gene expression profiles clearly distinguish samples from the three groups (FIG. 1A). Clustering resulted in two major classes of samples. All ipsilateral samples clustered in one class, and all sham controls in the other class. Interestingly, 3 of the 5 contralateral samples formed a subcluster in the ipsilateral class, whereas the other 2 contralateral samples clustered with sham samples. These data demonstrate that gene expression profiling can clearly distinguish operated joints from sham controls, whereas contralateral joints show higher variability. 3 out of 5 contralateral samples showed significantly different expression profiles compared to sham, indicating that contralateral joints are not an appropriate control.

Next, whether expression of known marker genes of OA is upregulated in the osteoarthritic model (FIG. 1B) was explored. The expression of proteases including a disintegrin-like and metalloproteinase with thrombospondin type 1 motif 5 (Adamts5) (23, 24), matrix metalloproteinase 2 (Mmp2) (25), and matrix metalloproteinase 13 (Mmp13) (26-28) increased in ipsilateral OA cartilage, similar to other described OA-related factors including chitinase 3-like 1 (Chi3l1, encoding cartilage glycoprotein 39) (29), prostaglandin E synthase (Ptges) (30), prostaglandin-endoperoxide synthase 2 (Ptgs2, encoding Cox2) (31) and transforming growth factor-beta 2 (Tgf-β2) (12).

Altered production of extracellular matrix (ECM) components is common in OA cartilage (32, 33) and was observed in ipsilateral cartilage samples (FIG. 1C). This included increases in several types of collagen, many of which (like type 1 collagen alpha-1 (Colla1)) are not expressed at high levels in healthy cartilage (34, 35) but were increased 2- to 4-fold in ipsilateral samples. Versican (Cspg2), lumican (Lum) and syndecan I (Sdcl) were other up-regulated ECM genes (FIG. 1C).

### Verification of Changes in Gene Expression by Real-Time PCR

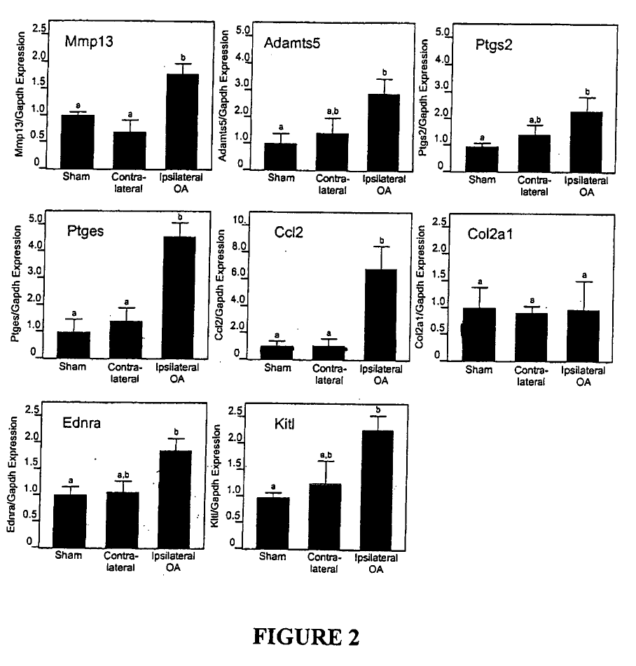

Independent animals were then used to confirm selected changes in gene expression by other approaches. Real-time PCR demonstrated increased expression of Mmp13, Adamts5, Ptgs2, Ptges, Ccl2, Ednra and Kitl in ipsilateral OA cartilage, thus confirming the microarray data (FIG. 2). Microarray analyses also demonstrated changes in the contralateral expression of some genes compared to sham controls. Although similar changes were observed with real-time PCR, no statistically significant differences were confirmed for the probes tested, in agreement with the heterogeneity of contralateral samples shown by unsupervised clustering. The expression of Collagen II (Col2a1) was analyzed because it was not included in the initial probe list due to substantial raw signal intensity variation between samples. Real-time PCR found similar variability between samples without statistically significant differences (FIG. 2). Overall, confirmation of the microarray expression patterns by real-time PCR indicates that the microarray data accurately reflects gene expression patterns.

### Confirmation of Gene Expression at the Protein Level

To validate the microarray gene expression data at the protein level and in vivo, the expression of MMP13 was examined using tissue sections of sham, contralateral and ipsilateral knees by immunofluorescence. Histological sections of articular cartilage from the medial compartment of sham, contralateral, and ipsilateral OA joints were processed and probed with anti-MMP13 antibodies followed by secondary antibodies conjugated to FITC. MMP13 expression was indicated by green fluorescence and nuclear counterstain with Toto-3 iodide was indicated in red. MMP13 protein expression was markedly increased in ipsilateral cartilage as shown by more intense signal in the region of the major cartilage surface defect, and detected throughout the cartilage interterritorial matrix, territorial matrix and chondrocyte lacunae, as compared to contralateral and sham cartilage.

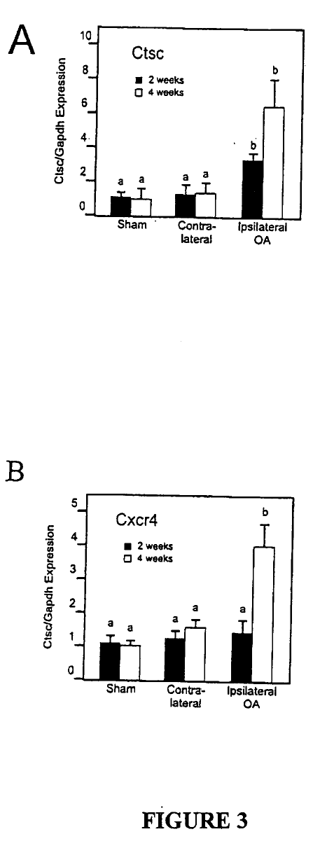

The spatial and temporal expression of cathepsin C (CTS-C) and chemokine (CXC) receptor 4 (CXCR4) was also examined by real-time PCR and immunostaining. Analysis of RNA samples isolated two weeks after surgery determined that Ctsc expression, but not Cxcr4 expression, increased in ipsilateral cartilage at this earlier time point (FIG. 3A/B). Both genes were increased in ipsilateral cartilage at the 4 week post-surgery time point, in agreement with the microarray data. Immunohistochemistry results confirmed the RNA expression data and determined that CTS-C expression increased in ipsilateral OA cartilage at 2, 4, and 8 weeks post surgery, compared to sham controls. However, CXCR4 expression did not increase until 4 weeks post-surgery, and appeared to increase further at the 8 week time point. Interestingly, CXCR4 expression was also increased in hypertrophic chondrocytes of the growth plates of all animals tested. These results further validate the microarray expression data, but also suggest differential temporal profiles for different genes identified in the arrays.

Values shown represent gene expression as determined by ACT analysis, normalized to GAPDH, and relative to Sham controls (set to 1). The mean (n=5) plus standard error of the mean (S.E.M.) is indicated and statistical significance is indicated by a different letter (a, b) when P<0.05. Immunohistochemistry was performed on histological sections from sham, contralateral, and ipsilateral OA articular knee joints at 2, 4, and 8 weeks post-surgery. Antibodies against cathepsin C (CTS-C) and chemokine (CXC) receptor 4 were used to assess the spatial and temporal expression of each protein by colourimetric detection (brown precipitate). All sections were counterstained with haematoxylin (blue stain).

### Analyzing Categories of Regulated Genes

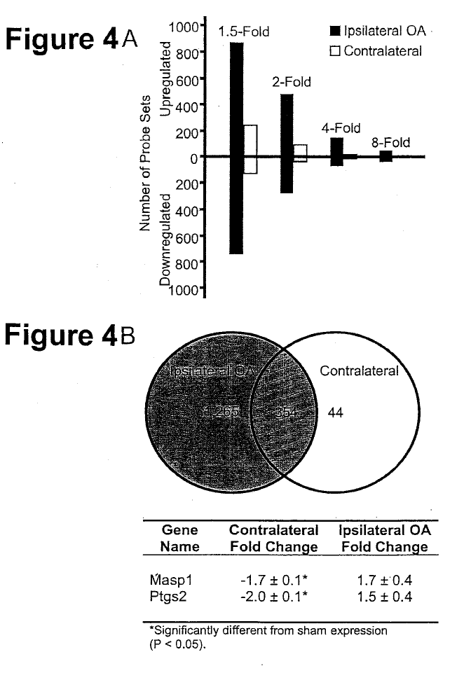

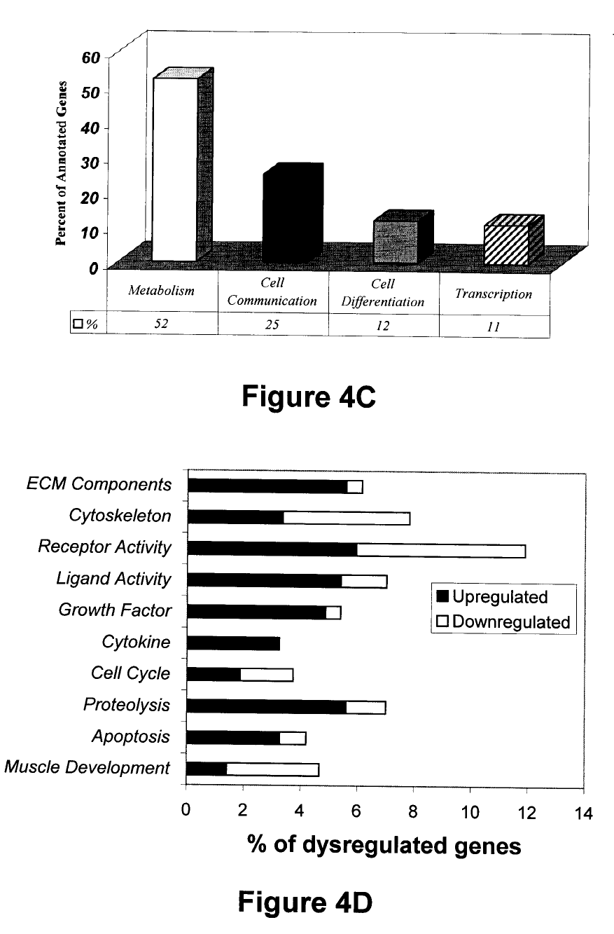

Having confirmed that known OA markers show the expected expression pattern in the present arrays and that alternative methods validate the array data, the present data sets were analyzed in more detail. Groups of differentially expressed transcripts in each treatment group were separated by setting minimum fold-change cutoffs. Comparison of ipsilateral cartilage to controls revealed 1,619, 722, 135, and 20 differentially expressed probe sets with at least 1.5-, 2-, 4-, and 8-fold changes, respectively (FIG. 4A). Comparison of contralateral cartilage to controls identified 398, 91, and 10 differentially expressed probes with at least 1.5-, 2-, and 4-fold changes (no gene displayed an 8-fold change in expression) (FIG. 4A). The identity of the genes in each group were then examined for overlap. Interestingly, 354 of the 398 transcripts that were differentially regulated in contralateral cartilage were also dysregulated in ipsilateral OA cartilage (FIG. 4B), the vast majority of which were dysregulated in the same direction (up or down). The only exceptions were Ptgs2 (encoding COX2) and Masp1, both of which were down-regulated in contralateral and up-regulated in ipsilateral cartilage. The full list of differentially expressed genes in both treatments is included below in Table 1.

TABLE 1
Fold changes in gene expression in contralateral and ipsilateral OA cartilage.

| | | | Ipsi- |
|---|---|---|---|
| | | Contra- | lateral |
| | | lateral | OA |
| Common | | Fold | Fold |
| Name | Description | Change | Change |
| 02-Sep | septin 2 | 1.0 | 1.3 |
| Abcc1 | ATP-binding cassette, sub-family C (CFTR/MRP), member 1 | 0.8 | 0.7 |
| Abcc9 | ATP-binding cassette, sub-family C (CFTR/MRP), member 9 | 1.2 | 1.6 |
| Abcd3 | ATP-binding cassette, sub-family D (ALD), member 3 | 0.8 | 0.7 |
| Abcg2 | UI-R-DR0-cjc-i-20-0-UI.s1 UI-R-DR0 Rattus norvegicus cDNA clone UI-R-DR0-cjc-i-20-0-UI 3′, mRNA sequence. | 1.1 | 1.3 |
| Acaa1 | acetyl-Coenzyme A acyltransferase 1 (peroxisomal 3-oxoacyl-Coenzyme A thiolase) | 0.7 | 0.6 |
| Acadl | acetyl-Coenzyme A dehydrogenase, long-chain | 0.9 | 0.7 |
| Ace | angiotensin 1 converting enzyme 1 | 0.9 | 0.6 |
| Ace | angiotensin 1 converting enzyme 1 | 0.7 | 0.5 |
| Aco2 | mitochondrial aconitase (nuclear aco2 gene) | 1.3 | 1.3 |
| Acox3 | acyl-Coenzyme A oxidase 3, pristanoyl | 0.9 | 1.2 |
| Acta1 | actin alpha 1 | 1.3 | 0.1 |
| Actn3 | actinin alpha 3 | 1.1 | 0.1 |
| Actn4 | actinin alpha 4 | 1.1 | 1.4 |
| Actr3 | actin-related protein 3 homolog (yeast) | 1.0 | 1.3 |
| Acvr1 | activin type I receptor | 0.9 | 1.1 |
| Acy1 | aminoacylase 1 | 0.7 | 0.5 |
| Ada | adenosine deaminase | 1.3 | 2.4 |
| Adam17 | a disintegrin and metalloproteinase domain 17 | 1.1 | 1.3 |
| Adamts5 | a disintegrin-like and metalloprotease (reprolysin type) with thrombospondin type 1 motif, 5 (aggrecanase-2) | 1.1 | 1.6 |
| Adh4 | alcohol dehydrogenase 4 (class II), pi polypeptide | 1.0 | 0.9 |
| Adn | adipsin | 4.1 | 1.6 |
| Adnp | activity-dependent neuroprotective protein | 0.9 | 0.9 |
| Adora1 | adenosine A1 receptor | 0.9 | 0.8 |
| Adprt | ADP-ribosyltransferase 1 | 1.2 | 1.1 |
| Agt | angiotensinogen | 0.8 | 0.5 |
| Agtr1a | angiotensin II receptor, type 1 (AT1A) | 1.5 | 2.9 |
| Agtr2 | Transcribed sequences | 1.3 | 0.7 |
| Ak3 | Rattus norvegicus adenylate kinase 3 (Ak3), mRNA. | 0.8 | 1.0 |
| Ak4 | adenylate kinase 4 | 0.7 | 0.8 |
| Akr7a2 | aldo-keto reductase family 7, member A2 (aflatoxin aldehyde reductase) | 1.2 | 1.5 |
| Alad | aminolevulinate, delta-, dehydratase | 0.9 | 0.6 |
| Aldh1a3 | aldehyde dehydrogenase family 1, subfamily A3 | 1.0 | 3.5 |
| Aldh3a2 | aldehyde dehydrogenase family 3, subfamily A2 | 1.0 | 0.9 |
| Aldh9a1 | DRNCBF10 Rat DRG Library Rattus norvegicus cDNA clone DRNCBF10 5′, mRNA sequence. | 0.9 | 0.8 |
| Aldoc | aldolase C, fructose-biphosphate | 0.7 | 0.6 |
| Amhr2 | anti-Mullerian hormone type 2 receptor | 1.0 | 0.6 |
| Amsh | associated molecule with the SH3 domain of STAM | 0.9 | 0.8 |
| Ania4 | | 1.2 | 2.7 |
| Ank | progressive ankylosis | 0.9 | 0.8 |
| Ank | progressive ankylosis | 0.9 | 0.7 |
| Ank | progressive ankylosis | 0.9 | 0.6 |
| Anpep | alanyl (membrane) aminopeptidase | 1.3 | 2.6 |
| Anxa2 | calpactin I heavy chain | 1.0 | 1.2 |
| Anxa6 | annexin VI | 1.0 | 0.8 |
| Anxa7 | annexin A7 | 0.8 | 0.8 |
| Apobec1 | apolipoprotein B editing complex 1 | 1.6 | 2.0 |
| App | amyloid beta (A4) precursor protein | 1.0 | 0.9 |
| App | amyloid beta (A4) precursor protein | 0.9 | 0.7 |
| App | amyloid beta (A4) precursor protein | 0.9 | 0.5 |
| Appils | leucyl-specific aminopeptidase PILS | 1.2 | 1.2 |
| Aqp1 | aquaporin 1 | 1.2 | 5.0 |
| Aqp3 | aquaporin 3 | 1.0 | 3.4 |
| Ar | androgen receptor | 0.8 | 0.5 |
| Arf2 | ADP-ribosylation factor 2 | 1.0 | 1.3 |
| Arf4 | ADP-ribosylation factor 4 | 1.1 | 1.2 |
| Arf5 | ADP-ribosylation factor 5 | 0.8 | 0.6 |
| Arf6 | ADP-ribosylation factor 6 | 1.3 | 2.0 |
| Arfd1 | ADP-ribosylation factor domain protein 1, 64 kD | 0.9 | 0.8 |
| Arg2 | arginase 2 | 1.0 | 0.8 |
| Argbp2 | Arg/Abl-interacting protein ArgBP2 | 1.1 | 0.8 |
| Arha2 | plysia ras-related homolog A2 | 1.1 | 1.3 |
| Arhgef5 | Rho guanine nucleotide exchange factor (GEF) 5 | 0.6 | 0.7 |
| Arhgef9 | DRNCLB01 Rat DRG Library Rattus norvegicus cDNA clone DRNCLB01 5′, mRNA sequence. | 0.9 | 0.5 |
| Arl3 | ADP-ribosylation-like 3 | 1.0 | 0.8 |
| Arl4 | ADP-ribosylation-like 4 | 1.3 | 1.5 |
| Arntl | aryl hydrocarbon receptor nuclear translocator-like | 5.2 | 5.0 |
| Arpp19 | cyclic AMP phosphoprotein, 19 kDa | 1.1 | 1.3 |
| Atp1a1 | ATPase, Na+K+ transporting, alpha 1 | 1.0 | 1.3 |
| Atp1b1 | ATPase Na+/K+ transporting beta 1 polypeptide | 1.1 | 1.5 |
| Atp1b1 | ATPase Na+/K+ transporting beta 1 polypeptide | 0.9 | 1.2 |
| Atp2a2 | ATPase, Ca++ transporting, cardiac muscle, slow twitch 2 | 1.3 | 1.7 |
| Atp2a2 | ATPase, Ca++ transporting, cardiac muscle, slow twitch 2 | 1.2 | 1.6 |
| Atp2b1 | ATPase, Ca++ transporting, plasma membrane 1 | 0.8 | 0.7 |
| Atp2b1 | ATPase, Ca++ transporting, plasma membrane 1 | 0.8 | 0.7 |
| Atp2b1 | UI-R-CA1-bbc-e-11-0-UI.s1 UI-R-CA1 Rattus norvegicus cDNA clone UI-R-CA1-bbc-e-11-0-UI 3′, mRNA sequence. | 0.7 | 0.7 |
| Atp2b1 | ATPase, Ca++ transporting, plasma membrane 1 | 0.7 | 0.6 |
| Atp2c1 | ATPase, Ca++-sequestering | 1.0 | 1.5 |
| Atp5d | ATP synthase, H+ transporting, mitochondrial F1 complex, delta subunit | 0.9 | 0.7 |
| Atp6b2 | ATPase, H+ transporting, lysosomal (vacuolar proton pump), beta 56/58 kDa, isoform 2 | 1.4 | 1.6 |
| Atp6b2 | ATPase, H+ transporting, lysosomal (vacuolar proton pump), beta 56/58 kDa, isoform 2 | 1.4 | 1.5 |
| Atp7b | ATPase, Cu++ transporting, beta polypeptide | 0.9 | 0.9 |
| Atp9a | ATPase, class II, type 9A | 1.0 | 0.6 |
| Avdp | androgen regulated vas deferens protein | 3.4 | 6.0 |
| AY228474 | DNA sequence AY228474 | 1.0 | 1.2 |
| Azgp1 | alpha-2-glycoprotein 1, zinc | 1.0 | 0.8 |
| B2m | beta-2 microglobulin | 1.4 | 1.4 |
| B4galt1 | UDP-Gal:betaGlcNAc beta 1,4-galactosyltransferase, polypeptide 1 | 0.9 | 1.5 |
| B4galt6 | go_component: Golgi apparatus [goid 0005794] [evidence IEA]; go_component: integral to membrane | 1.2 | 1.5 |
| | [goid 0016021] [evidence IEA]; go_function: magnesium ion binding [goid 0000287] [evidence IEA]; | | |
| | go_function: transferase activity [goid 0016740] [evidence IEA]; go_function: transferase | | |
| | activity, transferring glycosyl groups [goid 0016757] [evidence IEA]; go_function: | | |
| | manganese ion binding [goid 0030145] [evidence IEA]; go_process: carbohydrate metabolism [goid | | |
| | 0005975] [evidence IEA]; Rattus norvegicus UDP-Gal:betaGlcNAc beta 1,4-galactosyltransferase, | | |
| | polypeptide 6 (B4galt6), mRNA. | | |
| Bach | brain acyl-CoA hydrolase | 1.2 | 1.5 |
| Bambi | BMP and activin membrane-bound inhibitor, homolog (Xenopus laevis) | 1.5 | 0.9 |
| Basp1 | brain acidic membrane protein | 2.1 | 4.8 |
| Bcat2 | branched chain aminotransferase 2, mitochondrial | 0.6 | 0.3 |
| Bckdha | branched chain keto acid dehydrogenase subunit E1, alpha polypeptide | 0.9 | 0.7 |
| Bcl2 | B-cell leukemia/lymphoma 2 | 0.7 | 0.6 |
| Bcl2l1 | Bcl2-like 1 | 0.8 | 1.0 |
| Bet1 | blocked early in transport 1 homolog (S. cerevisiae) | 1.2 | 1.2 |
| Bgn | biglycan | 0.7 | 0.8 |
| Bhd | Transcribed sequence with weak similarity to protein ref: NP_495422.1 (C. elegans) F22D3.2.p [Caenorhabditis | 0.9 | 0.8 |
| | elegans] | | |
| Bhlhb2 | basic helix-loop-helix domain containing, class B2 | 0.5 | 0.7 |
| Bhlhb3 | basic helix-loop-helix domain containing, class B3 | 0.6 | 0.5 |
| BicD2 | UI-R-C0-jr-f-11-0-UI.s1 UI-R-C0 Rattus norvegicus cDNA clone UI-R-C0-jr-f-11-0-UI 3′, mRNA sequence. | 1.0 | 1.2 |
| Bin1 | myc box dependent interacting protein 1 | 0.8 | 0.6 |
| Bles03 | basophilic leukemia expressed protein BLES03 | 1.0 | 0.8 |
| Bmp2 | bone morphogenetic protein 2 | 1.2 | 1.1 |
| Bmp3 | bone morphogenetic protein 3 | 1.6 | 1.8 |
| Bmp4 | bone morphogenetic protein 4 | 0.9 | 0.6 |
| Bmp6 | bone morphogenetic protein 6 | 1.3 | 0.9 |
| Bmp6 | bone morphogenetic protein 6 | 1.1 | 0.7 |
| Brinp2 | BMP/retinoic acid-inducible neural-specific protein 2 | 1.1 | 1.2 |
| Bst1 | bone marrow stromal cell antigen 1 | 1.6 | 2.7 |
| Bub1b | budding uninhibited by benzimidazoles 1 homolog, beta (S. cerevisiae) | 1.8 | 1.8 |
| Bzrp | benzodiazepin receptor | 0.8 | 1.4 |
| C1s | complement component 1, s subcomponent | 1.1 | 2.8 |
| C2 | complement component 2 | 1.4 | 3.2 |
| C2 | complement component 2 | 1.3 | 3.2 |
| C2 | complement component 2 | 1.3 | 3.2 |
| C4a | palmitoyl-protein thioesterase 2 | 1.9 | 7.1 |
| C5r1 | complement component 5, receptor 1 | 1.3 | 1.9 |
| Ca3 | carbonic anhydrase 3 | 3.9 | 1.1 |
| Calcrl | calcitonin receptor-like | 1.4 | 3.2 |
| Calm1 | calmodulin 1 | 1.0 | 1.3 |
| Calm3 | calmodulin 3 | 1.0 | 0.8 |
| Camk1 | regulator of G-protein signalling 19 | 0.8 | 0.8 |
| Camk4 | calcium/calmodulin-dependent protein kinase IV | 0.7 | 0.4 |
| Capn6 | calpain 6 | 0.7 | 0.7 |
| Carhsp1 | calcium regulated heat stable protein 1 | 1.2 | 1.7 |
| CAR-XI | carbonic anhydrase-related XI protein | 0.9 | 0.7 |
| CAR-XI | carbonic anhydrase-related XI protein | 0.8 | 0.6 |
| Cask | calcium/calmodulin-dependent serine protein kinase | 0.9 | 1.2 |
| Casp12 | caspase 12 | 1.2 | 1.8 |
| Casp6 | caspase 6 | 1.0 | 1.5 |
| Casq2 | calsequestrin 2 | 1.1 | 3.2 |
| Casq2 | calsequestrin 2 | 1.1 | 2.2 |
| Cav | caveolin | 1.3 | 1.7 |
| Cav | caveolin | 1.2 | 1.6 |
| Cav | caveolin | 1.1 | 1.5 |
| Cav2 | Caveolin 2 (Cav2), mRNA | 1.1 | 1.4 |
| Cav2 | Caveolin 2 (Cav2), mRNA | 1.0 | 1.4 |
| Cav2 | Caveolin 2 (Cav2), mRNA | 1.0 | 1.4 |
| Cblb | Cas-Br-M (murine) ectropic retroviral transforming sequence b | 1.1 | 1.6 |
| Cbr4 | carbonyl reductase 4 | 1.0 | 0.7 |
| Cbx7 | chromobox 7 | 0.8 | 0.6 |
| Cck | cholecystokinin | 0.8 | 0.6 |
| Ccl2 | chemokine (C-C motif) ligand 2 | 1.8 | 18.8 |
| Ccna2 | cyclin A2 | 2.4 | 2.5 |
| Ccnb1 | cyclin B1 | 2.4 | 2.5 |
| Ccnb1 | cyclin B1 | 1.6 | 1.9 |
| Ccnd1 | cyclin D1 | 0.8 | 1.3 |
| Ccnd1 | cyclin D1 | 0.8 | 1.3 |
| Ccnd1 | cyclin D1 | 0.7 | 1.2 |
| Cd14 | CD14 antigen | 1.2 | 2.1 |
| Cd1d1 | CD1d1 antigen | 1.8 | 2.8 |
| Cd24 | CD24 antigen | 0.6 | 0.6 |
| Cd36 | synonym: Fat; CD36 antigen (collagen type I receptor thrombospondin receptor); fatty acid translocase; | 4.7 | 2.3 |
| | go_component: plasma membrane [goid 0005886] [evidence IDA]; go_component: integral to membrane [goid | | |
| | 0016021] [evidence TAS]; go_component: membrane [goid 0016020] [evidence IEA]; go_component: lysosome | | |
| | [goid 0005764] [evidence IEA]; go_function: fatty acid binding [goid 0005504] [evidence TAS]; go_function: | | |
| | receptor activity [goid 0004872] [evidence IGI]; go_function: cell adhesion molecule activity [goid 0005194] | | |
| | [evidence IEA]; go_process: fatty acid metabolism [goid 0006631] [evidence IDA]; | | |
| | go_process: long-chain fatty acid transport [goid | | |
| | 0015909] [evidence TAS]; go_process: transport [goid 0006810] [evidence IEA]; go_process: cell adhesion [goid | | |
| | 0007155] [evidence IEA]; Rattus norvegicus cd36 antigen (Cd36), mRNA. | | |
| Cd36 | FAT/CD36; Rattus norvegicus fatty acid translocase/CD36 mRNA, complete cds. | 2.5 | 1.5 |
| Cd36l2 | CD36 antigen (collagen type I receptor, thrombospondin receptor)-like 2 | 0.9 | 0.8 |
| Cd44 | CD44 antigen | 1.6 | 5.0 |
| Cd44 | CD44 antigen | 1.5 | 4.4 |
| Cd44 | CD44 antigen | 1.2 | 4.4 |
| Cd53 | CD53 antigen | 2.7 | 3.3 |
| Cd74 | CD74 antigen (invariant polpypeptide of major histocompatibility class II antigen-associated) | 2.1 | 2.4 |
| Cdc2a | cell division cycle 2 homolog A (S. pombe) | 1.6 | 2.1 |
| Cdc91l1 | CDC91 cell division cycle 91-like 1 (S. cerevisiae) | 1.0 | 0.9 |
| Cdh11 | cadherin-11 | 1.1 | 1.8 |
| Cdh13 | cadherin 13 | 1.0 | 5.1 |
| Cdh13 | cadherin 13 | 0.9 | 4.5 |
| Cdh2 | cadherin 2 | 1.7 | 2.1 |
| Cdk2 | cyclin dependent kinase 2 | 0.7 | 0.8 |
| Cdo1 | cytosolic cysteine dioxygenase 1 | 1.3 | 1.6 |
| Cdrap | cartilage derived retinoic acid sensitive protein | 1.1 | 0.7 |
| Cfl1 | cofilin 1 | 1.1 | 1.3 |
| Cgef2 | cAMP-regulated guanine nucleotide exchange factor II | 1.1 | 1.8 |
| Chek1 | checkpoint kinase 1 homolog (S. pombe) | 1.1 | 1.5 |
| Chi3l1 | chitinase 3-like 1 (cartilage glycoprotein-39) | 1.1 | 2.5 |
| Chn2 | chimerin (chimaerin) 2 | 0.8 | 0.5 |
| Chn2 | chimerin (chimaerin) 2 | 0.8 | 0.4 |
| CHOT1 | choline transporter | 1.1 | 1.5 |
| Chrd | chordin | 0.9 | 1.1 |
| Cirbp | cold inducible RNA-binding protein | 0.7 | 0.6 |
| Cirl2 | calcium-independent alpha-latrotoxin receptor homolog 2 | 1.1 | 1.7 |
| Cited2 | Cbp/p300-interacting transactivator, with Gul/Asp-rich carboxy-terminal domain, 2 | 0.9 | 0.5 |
| Cited2 | Cbp/p300-interacting transactivator, with Glu/Asp-rich carboxy-terminal domain, 2 | 0.8 | 0.4 |
| Ckb | creatine kinase, brain | 1.6 | 2.0 |
| Cklf1 | chemokine-like factor 1 | 2.0 | 2.1 |
| Cklf1 | chemokine-like factor 1 | 1.8 | 1.8 |
| Ckm | creatine kinase, muscle | 1.0 | 0.1 |
| Cktsf1b1 | cysteine knot superfamily 1, BMP antagonist 1 | 0.9 | 0.6 |
| Clasp2 | CLIP-associating protein 2 | 0.9 | 0.7 |
| Cldn1 | UI-R-C0-ha-d-06-0-UI.s1 UI-R-C0 Rattus norvegicus cDNA clone UI-R-C0-ha-d-06-0-UI 3′, mRNA sequence. | 2.7 | 1.2 |
| Cln2 | go_component: lysosome [goid 0005764] [evidence IEA]; go_function: serine-type endopeptidase activity [goid | 1.1 | 1.2 |
| | 0004252] [evidence IEA]; go_function: peptidase activity [goid 0008233] [evidence IEA]; go_function: hydrolase | | |
| | activity [goid 0016787] [evidence IEA]; go_function: tripeptidyl-peptidase I activity [goid 0019131] [evidence IEA]; | | |
| | Rattus norvegicus ceroid-lipofuscinosis, neuronal 2 (Cln2), mRNA. | | |
| Cngb1 | cyclic nucleotide-gated channel beta subunit 1 | 0.9 | 0.9 |
| Cnr1 | cannabinoid receptor 1 | 1.3 | 0.8 |
| Cntf | ciliary neurotropic factor | 1.1 | 1.5 |
| Col12a1 | procollagen, type XII, alpha 1 | 1.1 | 3.2 |
| Col12a1 | procollagen, type XII, alpha 1 | 0.9 | 3.2 |
| Col1a1 | collagen, type 1, alpha 1 | 1.4 | 3.8 |
| Col1a1 | collagen, type 1, alpha 1 | 1.2 | 2.1 |
| Col1a2 | procollagen, type I, alpha 2 | 1.2 | 1.7 |
| Col1a2 | procollagen, type I, alpha 2 | 1.1 | 1.5 |
| Col23a1 | UI-R-BO0-aig-b-09-0-UI.s1 UI-R-BO0 Rattus norvegicus cDNA clone UI-R-BO0-aig-b-09-0-UI 3′, mRNA sequence. | 0.9 | 0.8 |
| Col3a1 | collagen, type III, alpha 1 | 1.0 | 1.4 |
| Col5a1 | collagen, type V, alpha 1 | 0.9 | 1.9 |
| Col5a1 | collagen, type V, alpha 1 | 0.8 | 1.5 |
| Col5a2 | collagen, type V, alpha 2 | 1.0 | 1.4 |
| Col5a2 | collagen, type V, alpha 2 | 1.0 | 1.3 |
| Col5a3 | collagen, type V, alpha 3 | 1.0 | 2.2 |
| Coq7 | demethyl-Q 7 | 1.0 | 0.8 |
| Cox5a | cytochrome c oxidase, subunit Va | 1.2 | 1.1 |
| Crabp2 | cellular retinoic acid binding protein 2 | 1.2 | 14.8 |
| Crcp | calcitonin gene-related peptide-receptor component protein | 1.0 | 0.7 |
| Crem | cAMP responsive element modulator | 1.2 | 1.1 |
| Crip2 | cysteine-rich protein 2 | 0.9 | 1.2 |
| Crkas | v-crk-associated tyrosine kinase substrate | 0.7 | 0.6 |
| Crko | avian sarcoma virus CT10 (v-crk) oncogene homolog | 0.9 | 1.1 |
| Crmp4 | dihydropyrimidinase-like 3 | 1.5 | 3.1 |
| Crmp4 | dihydropyrimidinase-like 3 | 1.4 | 2.9 |
| Cryab | crystallin, alpha B | 0.9 | 0.6 |
| Cryac | UI-R-CS0-btv-c-04-0-UI.s1 UI-R-CS0 Rattus norvegicus cDNA clone UI-R-CS0-btv-c-04-0-UI 3′, mRNA sequence. | 0.9 | 1.4 |
| Crygc | crystallin, gamma C | 0.7 | 0.6 |
| Csp | cysteine string protein | 0.9 | 0.8 |
| Cspg2 | chondroitin sulfate proteoglycan 2 (versican) | 1.1 | 2.8 |
| Cspg2 | chondroitin sulfate proteoglycan 2 (versican) | 1.0 | 1.8 |
| Cspg2 | chondroitin Sulfate proteoglycan 2 (versican) | 1.0 | 1.7 |
| Cspg2 | chondroitin sulfate proteoglycan 2 (versican) | 0.9 | 1.3 |
| Csrp2 | cysteine and glycine-rich protein 2 | 1.4 | 6.2 |
| Cst3 | cystatin C | 1.1 | 0.9 |
| Cstn3 | calsyntenin 3 | 0.9 | 0.6 |
| Cth | CTL target antigen | 0.4 | 0.2 |
| Cthrc1 | collagen triple helix repeat containing 1 | 1.8 | 3.6 |
| Ctnnb1 | Myosin heavy chain mRNA, 3′ end | 1.5 | 0.1 |
| Ctsc | cathepsin C | 2.2 | 4.9 |
| Ctsc | cathepsin C | 1.8 | 4.2 |
| Ctsh | cathepsin H | 1.3 | 1.8 |
| Ctss | cathepsin S | 1.5 | 2.7 |
| Cttnb | cortactin isoform B | 0.9 | 1.2 |
| Cubn | cubilin (intrinsic factor-cobalamin receptor) | 0.9 | 0.3 |
| Cugbp2 | CUG triplet repeat, RNA-binding protein 2 | 1.4 | 1.5 |
| Cuta | Similar to divalent cation tolerant protein CUTA (LOC294288), mRNA | 1.0 | 0.9 |
| Cx3cl1 | chemokine (C—X3—C motif) ligand 1 | 0.8 | 0.8 |
| Cxcr4 | Chemokine receptor (LCR1) | 2.9 | 4.2 |
| Cxcr4 | Chemokine receptor (LCR1) | 2.1 | 3.3 |
| Cxcr4 | Chemokine receptor (LCR1) | 2.0 | 2.8 |
| Cyb5 | cytochrome b5 | 0.8 | 0.5 |
| Cybb | endothelial type gp91-phox gene | 1.9 | 2.8 |
| Cybb | endothelial type gp91-phox gene | 1.7 | 2.3 |
| Cycs | cytochrome c, somatic | 1.3 | 1.4 |
| Cycs | cytochrome c, somatic | 1.3 | 1.4 |
| Cyp26b1 | UI-R-BS2-bei-b-04-0-UI.s1 UI-R-BS2 Rattus norvegicus cDNA clone UI-R-BS2-bei-b-04-0-UI 3′, mRNA sequence. | 0.8 | 1.0 |
| Cyp26b1 | cytochrome P450, family 26, subfamily b, polypeptide 1 | 0.8 | 1.0 |
| Daf | decay-accelarating factor | 1.4 | 2.7 |
| Daf | | 1.1 | 2.1 |
| Dag1 | dystroglycan 1 | 0.8 | 0.7 |
| Dbp | D site albumin promoter binding protein | 0.1 | 0.1 |
| Dbt | dihydrolipoamide branched chain transacylase E2 | 0.6 | 0.6 |
| Dcamkl1 | activity and neurotransmitter-induced early gene protein 4 (ania-4) | 1.0 | 2.3 |
| Dci | dodecenoyl-coenzyme A delta isomerase | 0.8 | 0.6 |
| Dcn | decorin | 1.1 | 1.4 |
| Dd5 | progestin induced protein | 1.0 | 1.3 |
| Ddc | dopa decarboxylase | 1.3 | 0.9 |
| Ddit3 | DNA-damage inducible transcript 3 | 0.7 | 0.5 |
| Ddost | Similar to oligosaccharyltransferase (LOC313648), mRNA | 1.0 | 0.8 |
| Ddp2 | small zinc finger-like protein DDP2 | 1.1 | 1.0 |
| Ddt | D-dopachrome tautomerase | 0.8 | 0.6 |
| DdxI | nuclear RNA helicase, DECD variant of DEAD box family | 1.3 | 1.4 |
| Dgat2 | diacylglycerol O-acyltransferase homolog 2 (mouse) | 0.6 | 0.5 |
| Dgat2 | diacylglycerol O-acyltransferase homolog 2 (mouse) | 0.5 | 0.3 |
| Dhcr7 | 7-dehydrocholesterol reductase | 0.8 | 0.7 |
| Dig1 | dithiolethione-inducible gene-1 | 0.9 | 0.7 |
| Dkk3 | dickkopf homolog 3 (Xenopus laevis) | 0.8 | 1.2 |
| Dlc1 | rho GTPase activating protein 7 | 0.7 | 0.9 |
| Dlc2 | dynein light chain-2 | 0.8 | 0.6 |
| Dlx5 | distal-less homeobox 5 | 0.6 | 0.4 |
| Dnch1 | dynein, cytoplasmic, heavy chain 1 | 1.1 | 1.5 |
| Dncic1 | dynein, cytoplasmic, intermediate chain 1 | 0.8 | 0.5 |
| Dpm2 | dolichol-phosphate mannosyltransferase 2 | 0.9 | 0.8 |
| Dpp3 | dipeptidylpeptidase III | 1.0 | 1.3 |
| Dpysl2 | dihydropyrimidinase-like 2 | 0.9 | 1.3 |
| Drip78 | dopamine receptor interacting protein | 0.9 | 1.2 |
| Dtr | diphtheria toxin receptor | 1.4 | 2.9 |
| Ech1 | enoyl coenzyme A hydratase 1 | 1.0 | 0.7 |
| Echs1 | enoyl Coenzyme A hydratase, short chain 1 | 0.9 | 0.7 |
| Ecm1 | extracellular matrix protein 1 | 0.8 | 2.0 |
| Edg1 | endothelial differentiation sphingolipid G-protein-coupled receptor 1 | 1.8 | 4.5 |
| Edg5 | endothelial differentiation, sphingolipid G-protein-coupled receptor, 5 | 0.8 | 0.8 |
| Edg8 | sphingosine 1-phosphate receptor | 0.9 | 0.7 |
| Ednra | endothelin receptor type A | 1.0 | 2.4 |
| Ednra | endothelin receptor type A | 0.8 | 2.4 |
| Eef1d | translation elongation factor 1-delta subunit | 1.0 | 0.9 |
| Eef2k | eukaryotic elongation factor-2 kinase | 0.7 | 1.1 |
| Eef2k | eukaryotic elongation factor-2 kinase | 0.7 | 1.0 |
| Efg | G elongation factor | 1.2 | 1.3 |
| Egf | epidermal growth factor | 0.7 | 0.5 |
| Egfl3 | MEGF6 | 1.0 | 2.0 |
| Egfr | epidermal growth factor receptor | 1.2 | 1.3 |
| Egln1 | EGL nine homolog 1 (C. elegans) | 0.8 | 0.9 |
| Egln3 | EGL nine homolog 3 (C. elegans) | 0.7 | 1.4 |
| Egr1 | early growth response 1 | 0.8 | 0.5 |
| Ehd4 | pincher | 1.2 | 1.5 |
| Eif4e | eukaryotic translation initiation factor 4E | 1.1 | 1.2 |
| Eif4g2 | eukaryotic translation initiation factor 4 gamma, 2 | 1.1 | 1.6 |
| Eif4g2 | eukaryotic translation initiation factor 4 gamma, 2 | 1.0 | 1.1 |
| Eif5 | eukaryotic initiation factor 5 (elF-5) | 0.9 | 1.0 |
| Elf1 | E74-like factor 1 (ets domain transcription factor) | 1.0 | 1.3 |
| Emp1 | epithelial membrane protein 1 | 1.0 | 1.3 |
| Enh | enigma homolog | 0.7 | 1.2 |
| Enigma | enigma (LIM domain protein) | 1.1 | 1.6 |
| Eno2 | enolase 2, gamma | 0.6 | 0.8 |
| Enpep | Similar to Fish protein (LOC309460), mRNA | 0.9 | 1.6 |
| Enpep | aminopeptidase A | 0.8 | 1.0 |
| Entpd1 | ectonucleoside triphosphate diphosphohydrolase 1 | 1.3 | 4.5 |
| Epas1 | endothelial PAS domain protein 1 | 0.8 | 0.6 |
| Epb4.1l3 | erythrocyte protein band 4.1-like 3 | 1.0 | 2.3 |
| Ephx1 | epoxide hydrolase 1 | 0.4 | 0.2 |
| Erg | v-ets erythroblastosis virus E26 oncogene like (avian) | 1.1 | 0.9 |
| Esm1 | endothelial cell-specific molecule 1 | 1.4 | 3.3 |
| Etl | ETL protein | 1.6 | 5.1 |
| Ets1 | v-ets erythroblastosis virus E26 oncogene homolog 1 (avian) | 0.9 | 1.1 |
| Ets2 | v-ets erythroblastosis virus E26 oncogene homolog 2 (avian) | 1.2 | 1.2 |
| F2r | coagulation factor II receptor | 1.1 | 2.3 |
| F3 | coagulation factor 3 | 1.1 | 9.8 |
| F8 | coagulation factor VIII | 1.2 | 1.8 |
| F8 | coagulation factor VIII | 1.2 | 1.7 |
| F8 | coagulation factor VIII | 1.1 | 1.4 |
| Fabp4 | fatty acid binding protein 4 | 6.0 | 1.0 |
| Fabp5 | fatty acid binding protein 5, epidermal | 1.0 | 2.4 |
| Facl6 | fatty acid Coenzyme A ligase, long chain 6 | 0.7 | 0.3 |
| Fads1 | fatty acid desaturase 1 | 1.0 | 2.1 |
| Fap | fibroblast activation protein | 1.0 | 1.2 |
| Fat | FAT tumor suppressor (Drosophila) homolog | 1.1 | 1.6 |
| Fau | Finkel-Biskis-Reilly murine sarcoma virusubiquitously expressed | 1.0 | 0.9 |
| Fbln5 | fibulin 5 | 0.7 | 0.5 |
| Fbp1 | fructose-1,6-biphosphatase 1 | 1.3 | 0.5 |
| Fbxo11 | F-box only protein 11 | 0.8 | 0.8 |
| Fcgr3 | Fc receptor, IgG, low affinity III | 1.7 | 3.0 |
| Fcgr3 | Fc receptor, IgG, low affinity III | 1.4 | 2.8 |
| Fcgrt | Fc receptor, IgG, alpha chain transporter | 0.9 | 0.8 |
| Fgfr2 | fibroblast growth factor receptor 2 | 0.9 | 0.4 |
| Fgl2 | fibrinogen-like 2 | 1.4 | 1.5 |
| Fhl2 | four and a half LIM domains 2 | 0.8 | 1.8 |
| Frag1 | FGF receptor activating protein 1 | 0.8 | 0.7 |
| Freq | frequenin homolog (Drosophila) | 0.7 | 0.8 |
| Frk | src related tyrosine kinase | 1.1 | 1.8 |
| Fstl | DRABXE03 Rat DRG Library Rattus norvegicus cDNA clone DRABXE03 5′, mRNA sequence. | 0.9 | 1.3 |
| Fstl3 | follistatin-like 3 | 0.8 | 1.1 |
| Fth1 | ferritin, heavy polypeptide 1 | 1.0 | 0.9 |
| Fut11 | alpha3-fucosyltransferase 11 | 0.8 | 0.9 |
| Fut4 | alpha 1,3-fucosyltransferase Fuc-T (similar to mouse Fut4) | 0.8 | 0.5 |
| Fxc1 | fractured callus expressed transcript 1 | 0.9 | 0.7 |
| Fxyd2 | FXYD domain-containing ion transport regulator 2 | 0.9 | 1.8 |
| Fxyd3 | FXYD domain-containing ion transport regulator 3 | 1.2 | 0.6 |
| Fyn | fyn proto-oncogene | 0.8 | 1.1 |
| Fzd1 | Transcribed sequence with strong similarity to protein ref: NP_003496.1 (H. sapiens) frizzled 1; frizzled | 1.0 | 2.0 |
| G3bp | Ras-GTPase-activating protein SH3-domain binding protein | 1.1 | 1.3 |
| G6pc | glucose-6-phosphatase, catalytic | 0.9 | 0.9 |
| G6pdx | glucose-6-phosphate dehydrogenase | 1.2 | 1.3 |
| Gabarap | gamma-aminobutyric acid receptor associated protein | 1.1 | 0.9 |
| Gabbr1 | gamma-aminobutyric acid (GABA) B receptor, 1 | 1.1 | 1.8 |
| Gadd45a | growth arrest and DNA-damage-inducible 45 alpha | 1.3 | 2.4 |
| Gak | cyclin G-associated kinase | 0.9 | 1.0 |
| Galnt1 | polypeptide GalNAc transferase T1 | 1.4 | 3.3 |
| Galnt1 | polypeptide GalNAc transferase T1 | 1.1 | 2.3 |
| Gap43 | growth associated protein 43 | 1.0 | 7.9 |
| Gas5 | Gas-5 growth arrest homolog non-translated mRNA sequence | 1.0 | 0.8 |
| Gas6 | growth arrest specific 6 | 0.8 | 0.5 |
| Gas7 | growth arrest specific 7 | 1.3 | 2.3 |
| Gbp | UI-R-CT0-buo-d-03-0-UI.s1 UI-R-CT0 Rattus norvegicus cDNA clone UI-R-CT0-buo-d-03-0-UI 3′, mRNA sequence. | 1.3 | 2.4 |
| Gbp2 | guanylate binding protein 2, interferon-inducible | 1.5 | 2.2 |
| Gch | GTP cyclohydrolase 1 | 0.9 | 1.3 |
| Gclc | glutamate-cysteine ligase catalytic subunit | 1.0 | 1.3 |
| Gcsh | glycine cleavage system protein H (aminomethyl carrier) | 1.1 | 0.8 |
| Gdi3 | guanosine diphosphate dissociation inhibitor 3 | 1.0 | 1.1 |
| Gfra2 | glial cell line derived neurotrophic factor family receptor alpha 2 | 1.0 | 1.1 |
| Ggh | gamma-glutamyl hydrolase | 1.2 | 0.7 |
| Ghr | growth hormone receptor | 1.0 | 0.5 |
| Ghr | growth hormone receptor | 0.8 | 0.5 |
| Giot1 | gonadotropin inducible ovarian transcription factor 1 | 0.9 | 0.8 |
| Gja1 | gap junction membrane channel protein alpha 1 | 1.0 | 1.8 |
| Gja1 | gap junction membrane channel protein alpha 1 | 0.9 | 1.6 |
| Gja4 | gap junction membrane channel protein alpha 4 | 1.4 | 1.8 |
| glb | diacetyl/L-xylulose reductase | 0.8 | 0.6 |
| Glb1 | galactosidase, beta 1 | 0.9 | 1.1 |
| Gli | GLI-Kruppel family member GLI | 0.9 | 0.7 |
| Glud1 | glutamate dehydrogenase 1 | 0.8 | 1.2 |
| Gmeb2 | glucocorticoid modulatory element binding protein 2 | 1.0 | 0.8 |
| Gmpr | guanosine monophosphate reductase | 0.9 | 0.4 |
| Gnai1 | guanine nucleotide binding protein, alpha inhibiting 1 | 0.8 | 0.6 |
| Gnaq | heterotrimeric guanine nucleotide-binding protein alpha q subunit | 1.0 | 1.2 |
| Gng5 | G protein gamma-5 subunit | 1.1 | 1.1 |
| Gosr2 | golgi SNAP receptor complex member 2 | 1.2 | 1.3 |
| Gosr2 | golgi SNAP receptor complex member 2 | 1.2 | 1.1 |
| Gp1bb | glycoprotein Ib (platelet), beta polypeptide | 0.7 | 0.7 |
| Gp38 | glycoprotein 38 | 0.9 | 1.3 |
| Gpc1 | glypican 1 | 1.1 | 2.0 |
| Gpc2 | cerebroglycan | 0.9 | 1.2 |
| Gpi | glucose phosphate isomerase | 0.7 | 0.8 |
| Gpm6b | glycoprotein m6b | 0.9 | 3.5 |
| Gpm6b | glycoprotein m6b | 0.8 | 1.9 |
| Gpr37l1 | Osteotesticular phosphatase | 0.6 | 0.2 |
| Gpr48 | UI-R-CV2-cid-a-09-0-UI.s1 UI-R-CV2 Rattus norvegicus cDNA clone UI-R-CV2-cid-a-09-0-UI 3′, mRNA sequence. | 1.0 | 0.8 |
| Gpr48 | G protein-coupled receptor 48 | 0.9 | 0.6 |
| Gpr64 | G protein-coupled receptor 64 | 0.9 | 0.6 |
| Gpx3 | glutathione peroxidase 3 | 1.5 | 1.5 |
| Grina | NMDA receptor glutamate-binding chain | 0.9 | 1.2 |
| GST13-13 | glutathione S-transferase, mitochondrial | 1.0 | 0.8 |
| Gstm1 | glutathione S-transferase, mu 1 | 0.8 | 0.6 |
| Gstm3 | glutathione S-transferase, mu type 3 (Yb3) | 1.0 | 0.4 |
| Gstm5 | glutathione S-transferase, mu 5 | 0.9 | 0.6 |
| Gstp2 | glutathione S-transferase, pi 2 | 0.7 | 0.4 |
| Gt198 | nuclear receptor coactivator GT198 | 1.0 | 0.8 |
| Gtf2ird1 | general transcription factor II I repeat domain-containing 1 | 1.2 | 1.3 |
| Gucy1a3 | guanylate cyclase 1, soluble, alpha 3 | 1.6 | 2.2 |
| Gucy1b3 | guanylate cyclase 1, soluble, beta 3 | 2.0 | 5.2 |
| Gucy1b3 | guanylate cyclase 1, soluble, beta 3 | 1.9 | 3.9 |
| H1f0 | H1 histone family, member 0 | 0.8 | 0.7 |
| H2a | Similar to Histone H2A.o (H2A/o) (H2A.2) (H2a-615) (LOC365877), mRNA | 0.9 | 0.7 |
| Has2 | hyaluronan synthase 2 | 0.7 | 0.6 |
| Hdgfrp3 | hepatoma-derived growth factor, related protein 3 | 1.9 | 2.6 |
| Herpud1 | homocysteine-inducible, endoplasmic reticulum stress-inducible, ubiquitin-like domain member 1 | 0.6 | 0.5 |
| Hexa | hexosaminidase A | 0.9 | 0.7 |
| Hey1 | hairy/enhancer-of-split related with YRPW motif 1 | 1.6 | 2.1 |
| Hfe | hemochromatosis | 0.8 | 0.5 |
| Hig1 | hypoxia induced gene 1 | 0.8 | 0.9 |
| Hig1 | hypoxia induced gene 1 | 0.7 | 0.8 |
| Hint4 | histidine triad nucleotide binding protein 4 | 0.9 | 0.7 |
| Hmgcr | 3-hydroxy-3-methylglutaryl-Coenzyme A reductase | 1.0 | 1.3 |
| Hmox1 | synonyms: Ho1, Heox, Hmox, Ho-1, HEOXG; Heme oxygenase; Heme oxygenase 1; Rattus norvegicus heme | 1.1 | 2.9 |
| | oxygenase 1 (Hmox1), mRNA. | | |
| Hnf3b | hepatocyte nuclear factor 3, beta | 0.8 | 0.4 |
| hnRNPA3 | Similar to misshapen/NIK-related kinase isoform 2; GCK family kinase MINK; serine/threonine protein kinase | 1.1 | 1.1 |
| | (LOC294917), mRNA | | |
| Hnrpa1 | heterogeneous nuclear ribonucleoprotein A1 | 1.0 | 1.2 |
| Homer1 | homer, neuronal immediate early gene, 1 | 0.9 | 0.8 |
| Hrasls3 | HRAS like suppressor | 1.0 | 0.6 |
| Hrmt1l2 | heterogeneous nuclear ribonucleoproteins methyltransferase-like 2 (S. cerevisiae) | 1.0 | 0.8 |
| Hs3st1 | heparan sulfate (glucosamine) 3-O-sulfotransferase 1 | 0.6 | 0.1 |
| Hsd17b10 | hydroxysteroid (17-beta) dehydrogenase 10 | 0.8 | 0.7 |
| Hsd17b7 | hydroxysteroid dehydrogenase 17 beta, type 7 | 0.8 | 1.2 |
| Hsd17b8 | Similar to KE6a (LOC361802), mRNA | 0.9 | 1.1 |
| Hsj2 | DnaJ-like protein | 1.2 | 1.4 |
| Hspa1a | heat shock 70 kD protein 1A | 0.6 | 1.5 |
| Hspa1a | heat shock 70 kD protein 1A | 0.6 | 1.3 |
| Hspb1 | synonym: Hsp27; This sequence comes from FIG. 1; Heat shock 27 kDa protein; heat shock 27 kDa protein 1; Rattus | 0.6 | 0.4 |
| | norvegicus heat shock 27 kDa protein 1 (Hspb1), mRNA. | | |
| Hspca | Transcribed sequence with moderate similarity to protein sp: P07900 (H. sapiens) HS9A_HUMAN Heat shock protein | 1.1 | 1.1 |
| | HSP 90-alpha | | |
| Htatip | HIV-1 Tat interactive protein, 60 kD | 0.9 | 0.6 |
| Htr5b | 5-hydroxytryptamine (serotonin) receptor 5B | 0.7 | 0.6 |
| Hyal2 | hyaluronidase 2 | 0.9 | 0.7 |
| IAG2 | implantation-associated protein | 1.3 | 1.3 |
| Ian1 | immune-associated nucleotide 1 | 1.5 | 2.2 |
| Ian4l1 | immune associated nucleotide 4 like 1 (mouse) | 2.0 | 3.6 |
| Ica1 | islet cell autoantigen 1, 69 kDa | 0.9 | 0.5 |
| Ick | intestinal cell kinase | 0.8 | 0.7 |
| Idb4 | inhibitor of DNA binding 4 | 0.8 | 0.4 |
| Idb4 | inhibitor of DNA binding 4 | 0.8 | 0.4 |
| Idb4 | inhibitor of DNA binding 4 | 0.7 | 0.3 |
| Idb4 | inhibitor of DNA binding 4 | 0.7 | 0.3 |
| Idb4 | inhibitor of DNA binding 4 | 0.6 | 0.2 |
| Idh3a | isocitrate dehydrogenase 3 (NAD+) alpha | 1.3 | 1.6 |
| Idi1 | isopentenyl-diphosphate delta isomerase | 1.2 | 1.9 |
| Idi1 | isopentenyl-diphosphate delta isomerase | 0.9 | 1.5 |
| Ier5 | RM5 mRNA for Ier5, partial sequence | 1.9 | 3.2 |
| Ifitm3l | interferon induced transmembrane protein 3-like | 1.5 | 3.5 |
| Igf1 | insulin-like growth factor 1 | 2.0 | 6.7 |
| Igf2 | insulin-like growth factor 2 | 0.9 | 0.6 |
| Igf2r | insulin-like growth factor 2 receptor | 1.1 | 1.5 |
| Igf2r | insulin-like growth factor 2 receptor | 0.9 | 1.3 |
| Igfbp3 | insulin-like growth factor binding protein 3 | 1.4 | 4.2 |
| Igfbp6 | insulin-like growth factor binding protein 6 | 1.0 | 2.9 |
| Igfbp6 | insulin-like growth factor binding protein 6 | 0.9 | 2.8 |
| Igsf6 | immunoglobulin superfamily, member 6 | 2.2 | 2.3 |
| Ihpk1 | inositol hexaphosphate kinase 1 | 0.9 | 0.8 |
| Il11ra1 | interleukin 11 receptor, alpha chain 1 | 1.0 | 0.7 |
| Il1r2 | interleukin 1 receptor, type II | 1.2 | 0.3 |
| Il2rg | interleukin 2 receptor, gamma chain | 2.2 | 4.0 |
| Impa2 | inositol (myo)-1(or 4)-monophosphatase 2 | 0.9 | 0.7 |
| Inhba | inhibin beta-A | 1.9 | 8.4 |
| Ipmk | inositol polyphosphate multikinase | 0.8 | 0.8 |
| Irs3 | insulin receptor substrate 3 | 1.1 | 0.6 |
| Itga6 | UI-R-BJ1-avd-c-01-0-UI.s1 UI-R-BJ1 Rattus norvegicus cDNA clone UI-R-BJ1-avd-c-01-0-UI 3′, mRNA sequence. | 0.9 | 0.8 |
| Itga6 | UI-R-E1-ft-c-04-0-UI.s1 UI-R-E1 Rattus norvegicus cDNA clone UI-R-E1-ft-c-04-0-UI 3′, mRNA sequence. | 0.8 | 0.6 |
| Itgb1 | integrin beta 1 | 1.0 | 1.2 |
| Itpr1 | | 1.0 | 0.8 |
| Itpr2 | inositol 1,4,5-triphosphate receptor 2 | 0.9 | 0.9 |
| Itpr2 | inositol 1,4,5-triphosphate receptor 2 | 0.9 | 0.8 |
| Itpr3 | inositol 1,4,5-triphosphate receptor 3 | 0.7 | 0.8 |
| Ivd | isovaleryl coenzyme A dehydrogenase | 0.8 | 0.6 |
| Jag1 | jagged 1 | 0.9 | 1.5 |
| Jak2 | Janus kinase 2 | 1.0 | 1.2 |
| Jdp1 | Jun dimerization protein 1 gene | 1.1 | 0.7 |
| Jup | junction plakoglobin | 1.1 | 1.4 |
| Kcnj11 | potassium inwardly rectifying channel, subfamily J, member 11 | 0.9 | 0.8 |
| Kcnn1 | potassium intermediate/small conductance calcium-activated channel, subfamily N, member 1 | 1.0 | 0.9 |
| Kcnn2 | potassium intermediate/small conductance calcium-activated channel, subfamily N, member 2 | 0.8 | 0.6 |
| Kdr | kinase insert domain protein receptor | 1.4 | 3.5 |
| Kif2 | Similar to RIKEN cDNA 1500031M22 (LOC294718), mRNA | 1.2 | 1.1 |
| Kif3c | kinesin family member 3C | 1.4 | 1.3 |
| Kif5b | kinesin family member 5B | 1.2 | 1.8 |
| Kif5b | kinesin family member 5B | 1.1 | 1.3 |
| Kitl | kit ligand | 2.1 | 5.3 |
| Klf15 | Kruppel-like factor 15 | 0.7 | 0.3 |
| Klf4 | Kruppel-like factor 4 (gut) | 1.4 | 2.2 |
| Knsl1 | kinesin-like 1 | 1.7 | 2.0 |
| Kpna1 | karyopherin alpha 1 (importin alpha 5) | 1.1 | 1.2 |
| Kpna2 | karyopherin (importin) alpha 2 | 1.5 | 2.0 |
| Krim1 | KRAB box containing zinc finger protein | 1.1 | 0.8 |
| Krt1-18 | Similar to cytokeratin (LOC294853), mRNA | 0.8 | 0.4 |
| Krt2-8 | keratin complex 2, basic, gene 8 | 0.9 | 0.4 |
| Lamc1 | laminin, gamma 1 | 0.9 | 1.4 |
| Lap1c | lamina-associated polypeptide 1C | 1.0 | 0.8 |
| Lbp | lipopolysaccharide binding protein | 1.4 | 6.1 |
| Ldha | lactate dehydrogenase A | 1.0 | 1.1 |
| Ldhb | lactate dehydrogenase B | 1.1 | 0.6 |
| Ldlr | low density lipoprotein receptor | 1.0 | 1.7 |
| Lect1 | leukocyte cell derived chemotaxin 1 | 1.0 | 0.2 |
| Lepre1 | leprecan | 0.8 | 0.7 |
| Lfng | lunatic fringe gene homolog (Drosophila) | 0.9 | 0.8 |
| Lgals1 | lectin, galactose binding, soluble 1 | 1.3 | 3.5 |
| Lgals3 | lectin, galactose binding, soluble 3 | 0.9 | 1.5 |
| Lgl1 | late gestation lung protein 1 | 1.4 | 3.2 |
| Lgl1 | late gestation lung protein 1 | 1.2 | 2.5 |
| Lig1 | DNA ligase I | 1.0 | 0.8 |
| Lig1 | DNA ligase I | 0.9 | 0.8 |
| LOC170824 | tumor suppressor pHyde | 0.8 | 1.3 |
| LOC171161 | common salivary protein 1 | 0.7 | 0.0 |
| Loc192245 | heat shock 20-kDa protein | 0.8 | 0.6 |
| LOC246046 | liver regeneration p-53 related protein | 1.1 | 0.9 |
| LOC246266 | lysophospholipase | 1.4 | 3.2 |
| LOC246273 | neuronal cell death inducible putative kinase (NIPK); induced by NGF-depletion; Rattus sp. mRNA for | 0.9 | 0.7 |
| | kinase, complete cds. | | |
| LOC246307 | asparaginase-like sperm autoantigen | 1.0 | 0.8 |
| LOC246768 | cytosolic leucine-rich protein | 1.6 | 2.3 |
| LOC257646 | FERM-domain-containing protein 163SCII | 0.8 | 1.3 |
| LOC257646 | FERM-domain-containing protein 163SCII | 0.8 | 1.2 |
| LOC259246 | alpha-2u globulin PGCL5 | 0.5 | 0.3 |
| LOC260327 | peroxisomal protein | 0.7 | 0.6 |
| LOC286890 | tropomyosin isoform 6 | 0.8 | 0.7 |
| LOC286890 | tropomyosin isoform 6 | 0.7 | 0.7 |
| LOC286921 | aldose reductase-like protein | 1.1 | 3.1 |
| LOC287642 | galactose transporter | 1.3 | 1.3 |
| LOC289809 | putatative 28 kDa protein | 1.1 | 1.2 |
| LOC292624 | Similar to glioma tumor suppressor candidate region gene 2 (LOC292624), mRNA | 0.8 | 0.6 |
| LOC293589 | Similar to hypothetical protein BC004409 (LOC293589), mRNA | 1.1 | 0.8 |
| LOC296466 | BWK-1 | 1.0 | 0.8 |
| LOC296466 | BWK-1 | 0.9 | 0.8 |
| LOC296466 | BWK-1 | 0.8 | 0.7 |
| LOC298934 | androgen-responsive gene encoding an ARD-like protein | 1.1 | 0.8 |
| LOC301123 | Similar to RE70703p-like protein (LOC301123), mRNA | 0.9 | 0.7 |
| LOC304887 | Similar to Ral-A exchange factor RalGPS2 (LOC304887), mRNA | 1.3 | 0.8 |
| LOC306417 | putative scaffolding protein POSH | 0.8 | 0.7 |
| LOC316122 | Similar to CGI58 homolog (LOC316122), mRNA | 0.8 | 1.3 |
| LOC361537 | Similar to DAP12 (LOC361537), mRNA | 2.2 | 2.2 |
| LOC361873 | coxsackie-adenovirus receptor-like | 1.3 | 1.7 |
| LOC362246 | hypothetical protein | 1.0 | 1.2 |
| LOC54410 | alkaline phosphodiesterase | 1.2 | 3.9 |
| LOC60627 | component of rsec6/8 secretory complex p71 (71 kDa) | 1.2 | 1.8 |
| LOC64300 | C1-tetrahydrofolate synthase | 1.0 | 0.8 |
| LOC64312 | sperm membrane protein (YWK-II) | 0.7 | 0.7 |
| Loc65042 | tricarboxylate carrier-like protein | 0.7 | 0.8 |
| LOC81816 | ubiquitin conjugating enzyme | 1.0 | 0.8 |
| Lpl | lipoprotein lipase | 1.7 | 1.1 |
| Lrp16 | LRP16 protein | 0.8 | 0.5 |
| Lrp4 | low density lipoprotein receptor-related protein 4 | 1.5 | 2.3 |
| Lrpap1 | low density lipoprotein receptor-related protein associated protein 1 | 0.8 | 0.7 |
| Lsamp | limbic system-associated membrane protein | 0.9 | 0.3 |
| Ltbp1 | LanC (bacterial lantibiotic synthetase component C)-like 1 | 1.1 | 1.9 |
| Ltbp2 | latent transforming growth factor beta binding protein 2 | 1.1 | 2.1 |
| Lum | lumican | 1.3 | 2.0 |
| Luzp1 | leucine zipper protein 1 | 0.9 | 1.2 |
| Ly68 | lymphocyte antigen 68 | 2.2 | 6.3 |
| Ly68 | lymphocyte antigen 68 | 1.9 | 5.7 |
| Lyric | LYRIC | 0.9 | 1.2 |
| Lyz | Rat lysozyme gene, complete cds. | 1.7 | 1.8 |
| Madh3 | MAD homolog 3 (Drosophila) | 1.0 | 0.7 |
| Maged2 | melanoma antigen, family D, 2 | 0.9 | 0.7 |
| Maob | monoamine oxidase B | 1.2 | 1.9 |
| Map1b | microtubule-associated protein 1b | 1.5 | 1.0 |
| Map2k1 | mitogen activated protein kinase kinase 1 | 1.0 | 1.1 |
| Map2k2 | mitogen activated protein kinase kinase 2 | 1.4 | 1.4 |
| Map2k5 | mitogen activated protein kinase kinase 5 | 0.8 | 0.8 |
| Map2k6 | mitogen-activated protein kinase kinase 6 | 0.7 | 0.5 |
| Map3k12 | mitogen activated protein kinase kinase kinase 12 | 0.8 | 0.8 |
| Mapk6 | mitogen-activated protein kinase 6 | 1.0 | 1.2 |
| Mapk9 | stress activated protein kinase alpha II | 0.9 | 0.9 |
| Mapre1 | microtubule-associated protein, RP/EB family, member 1 | 1.3 | 1.4 |
| Masp1 | mannose-binding protein associated serine protease-1 | 0.6 | 1.7 |
| Mbtps1 | membrane-bound transcription factor protease, site 1 | 0.9 | 0.7 |
| Mcam | l-gicerin | 1.2 | 2.4 |
| Mepe | matrix extracellular phosphoglycoprotein with ASARM motif (bone) | 1.6 | 2.7 |
| Mfge8 | milk fat globule-EGF factor 8 protein | 1.1 | 0.8 |
| MGC72591 | Unknown (protein for MGC: 72591) | 0.8 | 0.7 |
| MGC72591 | Unknown (protein for MGC: 72591) | 0.8 | 0.7 |
| MGC72610 | Unknown (protein for MGC: 72610) | 0.8 | 0.8 |
| MGC72610 | Unknown (protein for MGC: 72610) | 0.8 | 0.8 |
| MGC72610 | Unknown (protein for MGC: 72610) | 0.8 | 0.7 |
| MGC72614 | Unknown (protein for MGC: 72614) | 1.7 | 3.5 |
| MGC72614 | Unknown (protein for MGC: 72614) | 1.4 | 3.3 |
| MGC72616 | Unknown (protein for MGC: 72616) | 0.7 | 0.7 |
| MGC72638 | Unknown (protein for MGC: 72638) | 1.0 | 0.6 |
| MGC72932 | Similar to NHP2-like protein 1 (High mobility group-like nuclear protein 2 homolog 1) ([U4/U6.U5] tri-snRNP | 1.2 | 1.2 |
| | 15.5 kDa protein) (OTK27) (LOC300092), mRNA | | |
| MGC72958 | MRNA for ribosomal protein L35 | 1.0 | 0.9 |
| MGC72996 | Unknown (protein for MGC: 72996) | 0.8 | 0.7 |
| MGC73002 | Unknown (protein for MGC: 73002) | 1.2 | 1.2 |
| MGEPS | Putative eps protein (MGEPS) mRNA, partial cds | 0.6 | 0.7 |
| Mgl | macrophage galactose N-acetyl-galactosamine specific lectin | 1.7 | 3.0 |
| Mgll | monoglyceride lipase | 0.9 | 0.6 |
| Mgmt | 0-6-methylguanine-DNA methyltransferase | 0.7 | 0.6 |
| Mgp | matrix Gla protein | 1.6 | 1.3 |
| Mgst1 | microsomal glutathione S-transferase 1 | 4.2 | 2.0 |
| Mitf | microphthalmia-associated transcription factor | 1.2 | 1.9 |
| Miz1 | Msx-interacting-zinc finger | 0.7 | 0.7 |
| Mlc3 | fast myosin alkali light chain | 1.5 | 0.0 |
| Mllt3 | myeloid/lymphoid or mixed-lineage leukemia (trithorax (Drosophila) homolog); translocated to, 3 | 0.8 | 0.6 |
| Mme | membrane metallo endopeptidase | 1.5 | 3.7 |
| Mmp12 | matrix metalloproteinase 12 | 0.8 | 7.5 |
| Mmp13 | matrix metalloproteinase 13 | 1.6 | 2.9 |
| Mmp14 | matrix metalloproteinase 14, membrane-inserted | 1.4 | 2.9 |
| Mmp16 | matrix metalloproteinase 16 | 1.0 | 1.2 |
| Mmp2 | matrix metalloproteinase 2 (72 KDa type IV collagenase) | 1.1 | 2.0 |
| Mmp24 | matrix metalloproteinase 24 (membrane-inserted) | 0.9 | 0.7 |
| Mmsdh | methylmalonate semialdehyde dehydrogenase gene | 0.8 | 0.5 |
| Mmsdh | methylmalonate semialdehyde dehydrogenase gene | 0.8 | 0.4 |
| Mor1 | malate dehydrogenase, mitochondrial | 1.2 | 1.1 |
| Mpeg1 | macrophage expressed gene 1 | 1.7 | 2.2 |
| Mpi | mannose phosphate isomerase | 1.3 | 1.5 |
| Mpst | mercaptopyruvate sulfurtransferase | 0.8 | 0.5 |
| Mrps18a | mitochondrial ribosomal protein S18A | 1.1 | 1.0 |
| Mrt1 | PDZ protein Mrt1 | 0.9 | 0.9 |
| Msn | moesin | 1.1 | 1.5 |
| Msx1 | homeo box, msh-like 1 | 0.9 | 1.5 |
| Mt1a | Metallothionein | 1.1 | 2.2 |
| Mterf | transcription termination factor, mitochondrial | 1.0 | 0.9 |
| Mtpn | myotrophin | 1.2 | 1.3 |
| Mtpn | myotrophin | 1.1 | 1.3 |
| Mtpn | myotrophin | 1.0 | 1.2 |
| Mug1 | alpha(1)-inhibitor 3, variant I | 1.1 | 0.6 |
| Mug1 | alpha(1)-inhibitor 3, variant I | 1.0 | 0.4 |
| Myadm | myeloid-associated differentiation marker | 0.7 | 1.2 |
| Mybbp1a | MYB binding protein 1a | 1.4 | 1.8 |
| Mybpc1 | myosin binding protein C, slow type | 0.8 | 0.3 |
| Mybph | norvegicus myosin binding protein H | 0.7 | 0.6 |
| Myh10 | myosin heavy chain 10, non-muscle | 1.1 | 0.8 |
| Myh9 | myosin, heavy polypeptide 9 | 1.1 | 1.8 |
| MYHC | type 2X myosin heavy chain | 0.9 | 0.1 |
| Myl2 | myosin, light polypeptide 2 | 1.2 | 0.1 |
| Myl3 | myosin, light polypeptide 3 | 0.5 | 0.3 |
| Myo1b | myosin Ib | 1.4 | 2.6 |
| Myo5a | myosin Va | 0.9 | 1.9 |
| Myoc | myocilin | 1.2 | 0.2 |
| Nap1l1 | nucleosome assembly protein 1-like 1 | 0.9 | 0.8 |
| Nap1l1 | nucleosome assembly protein 1-like 1 | 0.9 | 0.8 |
| Nap1l3 | nucleosome assembly protein 1-like 3 | 0.8 | 0.5 |
| Nbl1 | neuroblastoma, suppression of tumorigenicity 1 | 1.1 | 6.8 |
| Ncb5or | NADPH cytochrome B5 oxidoreductase | 1.0 | 1.2 |
| Ncoa1 | nuclear receptor coactivator 1 | 0.8 | 0.8 |
| Ncoa3 | nuclear receptor coactivator 3 | 0.7 | 0.7 |
| Ncstn | nicastrin | 0.8 | 0.9 |
| Ndn | necdin | 1.0 | 0.5 |
| Ndr4 | development-related protein | 1.0 | 1.8 |
| Ndrg2 | N-myc downstream-regulated gene 2 | 0.7 | 0.6 |
| Ndufa5 | NADH dehydrogenase (ubiquinone) 1 alpha subcomplex 5 | 1.0 | 1.0 |
| Nedd4a | neural precursor cell expressed, developmentally down-regulated gene 4A | 0.8 | 0.8 |
| Nedd4a | neural precursor cell expressed, developmentally down-regulated gene 4A | 0.6 | 0.6 |
| Nek6 | NIMA (never in mitosis gene a)-related expressed kinase 6 | 1.1 | 1.5 |
| Neurodap1 | protein carrying the RING-H2 sequence motif | 0.9 | 0.8 |
| Nfia | Transcribed sequence with strong similarity to protein sp: P00722 (E. coli) BGAL_ECOLI Beta-galactosidase | 0.8 | 0.7 |
| Nfia | nuclear factor I/A | 0.8 | 0.6 |
| Nfia | nuclear factor I/A | 0.6 | 0.6 |
| Nfib | nuclear factor I/B | 0.8 | 0.6 |
| Nfil3 | nuclear factor, interleukin 3, regulated | 1.4 | 2.5 |
| Nfkb1 | nuclear factor kappa B p105 subunit | 1.1 | 1.2 |
| Nid | nidogen (entactin) | 1.6 | 3.8 |
| Nid67 | putative small membrane protein NID67 | 1.2 | 0.8 |
| Nit1 | nitrilase 1 | 0.8 | 0.7 |
| Nme1 | expressed in non-metastatic cells 1 | 1.1 | 1.3 |
| Nme3 | expressed in non-metastatic cells 3, protein (nucleoside diphosphate kinase) | 0.9 | 0.7 |
| Nog | noggin | 1.3 | 0.8 |
| Nopp140 | nucleolar phosphoprotein p130 | 1.4 | 1.4 |
| Notch2 | notch gene homolog 2, (Drosophila) | 0.8 | 1.0 |
| Npap60 | nuclear pore associated protein | 0.8 | 0.7 |
| Npap60 | nuclear pore associated protein | 0.7 | 0.7 |
| Npl4 | homolog of yeast nuclear protein localization 4 | 1.0 | 1.2 |
| Npm1 | nucleophosmin 1 | 1.0 | 0.9 |
| Nptxr | neuronal pentraxin receptor | 0.8 | 0.8 |
| Nr1d1 | nuclear receptor subfamily 1, group D, member 1 | 0.4 | 0.3 |
| Nr1d2 | nuclear receptor subfamily 1, group D, member 2 | 0.5 | 0.4 |
| Nr2f2 | nuclear receptor subfamily 2, group F, member 2 | 1.1 | 2.0 |
| Nr3c1 | nuclear receptor subfamily 3, group C, member 1 | 0.8 | 0.8 |
| Nr3c2 | nuclear receptor subfamily 3, group C, member 2 | 0.8 | 0.6 |
| Nr4a1 | immediate early gene transcription factor NGFI-B | 0.9 | 0.6 |
| Nrbf2 | nuclear receptor binding factor 2 | 1.5 | 1.6 |
| Nrcam | Transcribed sequences | 1.5 | 0.7 |
| Nm | neuritin | 0.7 | 0.4 |
| Nrp | neuropilin | 1.7 | 2.2 |
| Nrp | neuropilin | 1.4 | 2.2 |
| Nrtn | neurturin | 0.7 | 0.4 |
| Ns5atp9 | Ns5atp9 protein | 2.4 | 2.1 |
| Nsep1 | nuclease sensitive element binding protein 1 | 0.9 | 0.9 |
| Ntel | NTE-related protein | 1.0 | 0.6 |
| Ntt73 | orphan transporter v7-3 | 0.6 | 0.9 |
| Nucb2 | NEFA precursor | 1.0 | 0.7 |
| Nudt2 | Transcribed sequence with weak similarity to protein sp: P50583 (H. sapiens) AP4A_HUMAN Bis | 0.9 | 0.7 |
| Nudt6 | antisense basic fibroblast growth factor | 0.8 | 0.5 |
| Nup54 | nucleoporin p54 | 0.7 | 0.7 |
| Ocln | occludin | 1.4 | 1.9 |
| Octn1 | organic cation transporter OCTN1 | 1.1 | 2.1 |
| Odf2 | sperm outer dense fiber major protein 2 | 1.2 | 1.4 |
| Ogfr | opioid growth factor receptor | 1.3 | 1.0 |
| Ol16 | LOC363049 (LOC363049), mRNA | 1.1 | 2.1 |
| Ol16 | visceral adipose tissue-specific transmembrane protein OL-16 | 0.9 | 1.7 |
| Oprs1 | opioid receptor, sigma 1 | 0.8 | 0.7 |
| Ostf1 | osteoclast stimulating factor 1 | 1.1 | 1.4 |
| Pabpn1 | poly(A) binding protein, nuclear 1 | 1.1 | 1.2 |
| Pace4 | Subtilisin-like endoprotease | 1.3 | 0.9 |
| Pafah1b1 | platelet-activating factor acetylhydrolase beta subunit (PAF-AH beta) | 1.0 | 1.2 |
| Pafah1b3 | platelet-activating factor acetylhydrolase, isoform 1b, alpha1 subunit | 0.7 | 0.5 |
| PAG608 | p53-activated gene 608 | 1.1 | 1.5 |
| Pak1 | p21 (CDKN1A)-activated kinase 1 | 1.2 | 1.7 |
| Pak2 | p21 (CDKN1A)-activated kinase 2 | 0.9 | 0.9 |
| Pam | peptidylglycine alpha-amidating monooxygenase | 1.0 | 0.8 |
| Panx3 | pannexin 3 | 1.9 | 4.1 |
| Parva | parvin, alpha | 0.8 | 1.0 |
| Pass1 | protein associating with small stress protein PASS1 | 0.9 | 0.7 |
| Pbef | pre-B-cell colony-enhancing factor | 1.0 | 1.3 |
| Pbp | phosphatidylethanolamine binding protein | 1.0 | 0.9 |
| Pck1 | UI-R-CY0-bxq-d-05-0-UI.s1 UI-R-CY0 Rattus norvegicus cDNA clone UI-R-CY0-bxq-d-05-0-UI 3′, | 1.9 | 1.2 |
| | mRNA sequence. | | |
| Pcmt1 | protein-L-isoaspartate (D-aspartate) O-methyltransferase 1 | 1.1 | 1.1 |
| Pcna | proliferating cell nuclear antigen | 1.3 | 1.3 |
| Pcolce | procollagen C-proteinase enhancer protein | 1.2 | 1.7 |
| Pcsk5 | proprotein convertase subtilisin/kexin type 5 | 0.9 | 1.6 |
| Pdcd2 | programmed cell death 2 | 0.9 | 0.8 |
| Pdcd4 | programmed cell death 4 | 0.7 | 0.5 |
| Pdcd6ip | hq35a05.x1 NCI_CGAP_Pr35 Rattus norvegicus cDNA clone IMAGE: 3121328 3′ similar to TR: O89014 | 1.0 | 1.3 |
| | O89014 ALIX-SF. [1];, mRNA sequence. | | |
| Pde2a | phosphodiesterase 2A, cGMP-stimulated | 1.5 | 1.4 |
| Pde8a | phosphodiesterase 8A | 1.1 | 3.0 |
| Pdgfra | platelet derived growth factor receptor, alpha polypeptide | 0.9 | 1.1 |
| Pdgfrb | platelet derived growth factor receptor, beta polypeptide | 0.9 | 1.9 |
| Pdgfrb | platelet derived growth factor receptor, beta polypeptide | 0.8 | 1.2 |
| Pdk1 | pyruvate dehydrogenase kinase 1 | 0.7 | 0.8 |
| Pdk2 | pyruvate dehydrogenase kinase 2 | 0.8 | 0.7 |
| Pdlim1 | PDZ and LIM domain 1 | 1.3 | 2.8 |
| Penk-rs | preproenkephalin, related sequence | 1.4 | 0.3 |
| Per2 | period homolog 2 | 0.3 | 0.3 |
| Per3 | period homolog 3 (Drosophila) | 0.4 | 0.3 |
| Pfn2 | profilin II | 0.9 | 0.8 |
| Pgam1 | phosphoglycerate mutase 1 | 1.0 | 1.0 |
| Pgrmc1 | progesterone receptor membrane component 1 | 0.9 | 0.7 |
| Phex | phosphate regulating gene with homologies to endopeptidases on the X chromosome | 1.6 | 2.4 |
| Phtf1 | putative homeodomain transcription factor 1 | 0.8 | 1.1 |
| Phyh | phytanoyl-CoA hydroxylase (Refsum disease) | 1.0 | 0.8 |
| Phyh | phytanoyl-CoA hydroxylase (Refsum disease) | 1.0 | 0.7 |
| Pias3 | protein inhibitor of activated STAT 3 | 0.7 | 0.6 |
| Pik3ca | phosphatidylinositol 3-kinase, catalytic, alpha polypeptide | 0.8 | 1.2 |
| Pim3 | serine threonine kinase pim3 | 0.7 | 0.6 |
| Pip5k2c | phosphatidylinositol-4-phosphate 5-kinase, type II, gamma | 0.8 | 0.8 |
| pips | Per1 interacting protein | 1.0 | 0.6 |
| Pitpn | phosphatidylinositol transfer protein | 1.1 | 1.3 |
| Pla2g4a | phospholipase A2, group IVA (cytosolic, calcium-dependent) | 0.9 | 1.4 |
| Plaur | Plasminogen activator, urokinase receptor | 1.1 | 5.1 |
| Plcb4 | phospholipase C, beta 4 | 1.3 | 1.3 |
| Pld1 | phospholipase D1 | 0.8 | 0.8 |
| Plec1 | plectin | 0.9 | 1.4 |
| Plg | plasminogen | 1.0 | 0.9 |
| Plk | polo-like kinase homolog (Drosophila) | 1.4 | 1.4 |
| Plp | proteolipid protein | 1.1 | 0.7 |
| Podxl | podocalyxin-like | 1.2 | 2.2 |
| Pou3f3 | POU domain, class 3, transcription factor 3 | 0.7 | 0.5 |
| Pp | pyrophosphatase | 1.3 | 1.3 |
| Ppap2a | phosphatidate phosphohydrolase type 2a | 0.9 | 1.4 |
| Ppap2c | phosphatidic acid phosphatase type 2c | 0.7 | 0.6 |
| Ppil3 | peptidylprolyl isomerase (cyclophilin)-like 3 | 1.2 | 1.3 |
| Ppp1r2 | protein phosphatase 1, regulatory (inhibitor) subunit 2 | 0.9 | 0.8 |
| Ppp2r1a | protein phosphatase 2 (formerly 2A), regulatory subunit A (PR 65), alpha isoform | 0.8 | 0.8 |
| Ppp2r2a | protein phosphatase 2 (formerly 2A), regulatory subunit B (PR 52), alpha isoform | 0.8 | 0.8 |
| Ppp3ca | protein phosphatase 3, catalytic subunit, alpha isoform | 1.0 | 1.4 |
| Ppp3ca | protein phosphatase 3, catalytic subunit, alpha isoform | 0.9 | 0.8 |
| Ppp3ca | protein phosphatase 3, catalytic subunit, alpha isoform | 0.9 | 0.8 |
| Ppp3cb | protein phosphatase 3, catalytic subunit, beta isoform | 0.9 | 1.1 |
| Ppp3cc | protein phosphatase 3 (formerly 2B), catalytic subunit, gamma isoform (calcineurin A gamma) | 0.9 | 0.8 |
| Ppp4c | protein phosphatase 4 (formerly X), catalytic subunit | 0.9 | 0.8 |
| Ppp4r1 | protein phosphatase 4, regulatory subunit 1 | 1.1 | 1.3 |
| Prdx3 | peroxiredoxin 3 | 1.3 | 1.1 |
| Prdx4 | peroxiredoxin 4 | 1.0 | 0.9 |
| Prkch | protein kinase C-eta | 1.8 | 1.8 |
| Prkwnk1 | protein kinase, lysine deficient 1 | 1.0 | 1.3 |
| Prkwnk4 | protein kinase, lysine deficient 4 | 0.8 | 0.7 |
| Pros1 | protein S | 1.0 | 1.5 |
| Prpsap2 | phosphoribosyl pyrophosphate synthetase-associated protein 2 | 0.9 | 0.7 |
| Prrx2 | paired related homeobox 2 | 1.0 | 3.1 |
| Psat1 | phosphoserine aminotransferase 1 | 1.4 | 2.7 |
| Psbp1 | prostatic steroid binding protein 1 | 1.5 | 0.4 |
| Pscd3 | pleckstrin homology, Sec7 and coiled/coil domains 3 | 1.0 | 1.3 |
| Psen2 | presenilin-2 | 0.8 | 0.8 |
| Psip2 | PC4 and SFRS1 interacting protein 2 | 0.7 | 0.7 |
| Psma3 | proteasome (prosome, macropain) subunit, alpha type 3 | 1.2 | 1.4 |
| Psma3 | proteasome (prosome, macropain) subunit, alpha type 3 | 1.0 | 1.3 |
| Pte1 | 4,8-dimethylnonanoyl-CoA thioesterase | 1.0 | 0.8 |
| Pte1 | 4,8-dimethylnonanoyl-CoA thioesterase | 0.8 | 0.7 |
| Pter | phosphotriesterase related | 1.3 | 1.6 |
| Ptgds | prostaglandin D2 synthase | 1.0 | 0.3 |
| Ptges | prostaglandin E synthase | 1.5 | 9.6 |
| Ptgfrn | prostaglandin F2 receptor negative regulator | 0.9 | 2.2 |
| Ptgis | prostaglandin I2 synthase | 0.8 | 0.6 |
| Ptgs2 | prostaglandin-endoperoxide synthase 2 | 0.5 | 1.5 |
| Ptp2E | protein tyrosine phosphatase 2E | 0.8 | 0.9 |
| Ptp4a1 | protein tyrosine phosphatase 4a1 | 1.3 | 1.5 |
| Ptpra | protein tyrosine phosphatase, receptor type, A | 1.0 | 0.9 |
| Ptprc | protein tyrosine phosphatase, receptor type, C | 1.7 | 2.2 |
| Ptprg | protein tyrosine phosphatase, receptor type, G | 0.9 | 1.6 |
| Ptprm | protein tyrosine phosphatase, receptor-type, M | 0.8 | 1.0 |
| Ptpro | protein tyrosine phosphatase, receptor type, O | 1.5 | 3.4 |
| Ptprr | protein tyrosine phosphatase, receptor type, R | 0.6 | 0.6 |
| Pxmp2 | peroxisomal membrane protein 2 | 0.9 | 0.7 |
| Pygl | liver glycogen phosphorylase | 0.6 | 0.6 |
| Pygm | muscle glycogen phosphorylase | 0.7 | 0.4 |
| Qdpr | quinoid dihydropteridine reductase | 0.9 | 0.8 |
| Rab10 | ras-related protein rab10 | 1.0 | 1.2 |
| Rab26 | RAB26, member RAS oncogene family | 0.8 | 0.7 |
| Rab38 | Rab38, member of RAS oncogene family | 2.0 | 2.7 |
| Rab8b | GTPase Rab8b | 1.1 | 1.3 |
| Rabggta | go_function: prenyltransferase activity [goid 0004659] [evidence IEA]; go_function: protein prenyltransferase activity | 1.0 | 0.8 |
| | [goid 0008318] [evidence IEA]; go_function: transferase activity [goid 0016740] [evidence IEA]; go_process: protein | | |
| | amino acid prenylation [goid 0018346] [evidence IEA]; Rattus norvegicus Rab geranylgeranyl transferase, a subunit | | |
| | (Rabggta), mRNA. | | |
| Ragb | GTP-binding protein ragB | 1.0 | 0.7 |
| Ralb | v-ral simian leukemia viral oncogene homolog B | 0.7 | 1.0 |
| Ralbp1 | ralA binding protein 1 | 1.0 | 0.7 |
| ram | low Mr GTP-binding protein | 1.4 | 1.3 |
| Ramp1 | receptor (calcitonin) activity modifying protein 1 | 1.2 | 0.8 |
| Ramp2 | receptor (calcitonin) activity modifying protein 2 | 1.1 | 0.4 |
| Ramp3 | receptor (calcitonin) activity modifying protein 3 | 0.5 | 0.2 |
| RAMP4 | ribosome associated membrane protein 4 | 1.3 | 1.4 |
| Rap1b | RAP1B, member of RAS oncogene family | 1.1 | 1.5 |
| Ratireb | iron-responsive element-binding protein | 0.7 | 0.7 |
| Rbl2 | retinoblastoma-like 2 | 0.7 | 0.5 |
| Rbp4 | retinol binding protein 4 | 1.1 | 0.7 |
| Rdbp | Similar to RD protein (WL623) (LOC294258), mRNA | 0.9 | 0.7 |
| Rdc1 | chemokine orphan receptor 1 | 1.3 | 2.1 |
| Rdh10 | retinol dehydrogenase 10 (all-trans) | 1.0 | 1.1 |
| re1 | epididymal secretory protein 1 | 1.1 | 1.4 |
| Rela | v-rel reticuloendotheliosis viral oncogene homolog A (avian) | 0.8 | 0.8 |
| Reln | reelin | 1.1 | 2.8 |
| rELO1 | fatty acid elongase 1 | 1.2 | 1.3 |
| rELO2 | fatty acid elongase 2 | 0.9 | 0.6 |
| rELO2 | fatty acid elongase 2 | 0.8 | 0.6 |
| rELO2 | fatty acid elongase 2 | 0.7 | 0.5 |
| Rfc2 | replication factor C (activator 1) 2 (40 kD) | 1.2 | 0.9 |
| Rgc32 | | 1.1 | 2.1 |
| Rgs10 | regulator of G-protein signaling 10 | 0.9 | 0.6 |
| Rgs19 | calcium/calmodulin-dependent protein kinase I | 1.0 | 0.9 |
| Rgs3 | regulator of G-protein signaling 3 | 1.3 | 1.1 |
| Rgs4 | regulator of G-protein signaling 4 | 1.4 | 6.8 |
| Rgs5 | regulator of G-protein signaling 5 | 1.8 | 10.0 |
| Rgs5 | regulator of G-protein signaling 5 | 1.3 | 7.0 |
| Rhk1 | KARP-1 binding protein 1 | 1.0 | 1.1 |
| Rhoip3 | Rho interacting protein 3 | 1.1 | 1.4 |
| Risc | retinoid-inducible serine caroboxypetidase | 1.0 | 1.6 |
| RNU28927 | GABA transporter | 0.6 | 0.7 |
| Rock2 | Rho-associated coiled-coil forming kinase 2 | 0.9 | 1.1 |
| Rpa2 | p32-subunit of replication protein A | 1.0 | 0.8 |
| Rpl10 | ribosomal protein L10 | 1.0 | 0.9 |
| Rpl17 | ribosomal protein L17 | 1.0 | 0.9 |
| Rpl17 | ribosomal protein L17 | 0.7 | 0.6 |
| Rpl19 | ribosomal protein L19 | 1.0 | 0.9 |
| Rpl21 | ribosomal protein L21 | 1.0 | 0.8 |
| Rpl22 | ribosomal protein L22 | 1.0 | 0.9 |
| Rpl24 | ribosomal protein L24 | 1.0 | 0.9 |
| Rpl27 | ribosomal protein L27 | 1.0 | 0.9 |
| Rpl28 | ribosomal protein L28 | 1.1 | 0.9 |
| Rpl28 | ribosomal protein L28 | 0.9 | 0.6 |
| Rpl29 | ribosomal protein L29 | 1.0 | 0.8 |
| Rpl31 | ribosomal protein L31 | 1.0 | 0.9 |
| Rpl36 | ribosomal protein L36 | 1.0 | 0.9 |
| Rpl37 | ribosomal protein L37 | 1.0 | 0.9 |
| Rpl4 | ribosomal protein L4 | 1.0 | 0.9 |
| Rpl7 | Similar to 60S RIBOSOMAL PROTEIN L7 (LOC301151), mRNA | 1.0 | 0.9 |
| Rps10 | ribosomal protein S10 | 1.0 | 0.8 |
| Rps12 | ribosomal protein S12 | 1.0 | 0.9 |
| Rps14 | ribosomal protein S14 | 1.0 | 0.9 |
| Rps15 | ribosomal protein S15 | 1.0 | 0.8 |
| Rps24 | Transcribed sequence with strong similarity to protein sp: P16632 (H. sapiens) RS24_HUMAN 40S ribosomal protein | 1.1 | 0.8 |
| | S24 | | |
| Rps25 | ribosomal protein s25 | 1.0 | 0.8 |
| Rps26 | ribosomal protein S26 | 1.0 | 0.8 |
| Rps27 | ribosomal protein S27 | 1.0 | 0.9 |
| Rps27a | ribosomal protein S27a | 0.9 | 0.7 |
| Rps4x | ribosomal protein S4, X-linked | 1.0 | 0.9 |
| Rps5 | ribosomal protein S5 | 1.1 | 0.9 |
| Rps7 | ribosomal protein S7 | 1.0 | 0.9 |
| RT1-Ba | butyrophilin-like 2 (MHC class II associated) | 1.3 | 1.4 |
| RT1-Da | Rat MHC class II RT1.u-D-alpha chain mRNA, 3′ end | 2.0 | 2.3 |
| RT1-Db1 | Rat MHC RT1 class II E-beta chain mRNA, 3′ end | 2.6 | 2.9 |
| RT1-DMb | major histocompatibility complex, class II, DM beta | 1.7 | 1.9 |
| Rtn1 | reticulon 1 | 0.7 | 0.4 |
| Rtp801 | HIF-1 responsive RTP801 | 1.5 | 1.9 |
| S100a1 | Similar to S-100 protein, alpha chain (LOC295214), mRNA | 1.1 | 0.7 |
| Sam68 | src associated in mitosis, 68 kDa | 0.8 | 0.8 |
| Sap1 | sodium channel associated protein 1 | 1.0 | 0.8 |
| Sat | spermidine/spermine N1-acetyl transferase | 1.0 | 1.3 |
| SC2 | synaptic glycoprotein SC2 | 1.1 | 0.8 |
| Sc4mol | sterol-C4-methyl oxidase-like | 1.1 | 1.3 |
| Sc5d | sterol-C5-desaturase (fungal ERG3, delta-5-desaturase)-like | 1.0 | 1.2 |
| Scap2 | src family associated phosphoprotein 2 | 1.3 | 1.9 |
| Scgf | stem cell growth factor | 0.8 | 1.3 |
| Scn1a | sodium channel, voltage-gated, type 1, alpha polypeptide | 0.6 | 0.4 |
| Scop | Circadian Oscillatory Protein (SCOP) | 0.9 | 0.9 |
| Scrg1 | scrapie responsive gene 1 | 1.0 | 0.8 |
| Sdc1 | UI-R-CV1-bsz-a-02-0-UI.s1 UI-R-CV1 Rattus norvegicus cDNA clone UI-R-CV1-bsz-a-02-0-UI 3′, mRNA sequence. | 1.3 | 2.6 |
| Sdc1 | syndecan 1 | 1.3 | 2.4 |
| Sdc2 | syndecan 2 | 1.1 | 1.3 |
| Sec61a | SEC61, alpha subunit (S. cerevisiae) | 1.0 | 1.3 |
| Sec61a | SEC61, alpha subunit (S. cerevisiae) | 1.0 | 1.3 |
| Selenbp2 | selenium binding protein 2 | 0.6 | 0.2 |
| Sema3a | Semaphorin 3a | 0.8 | 0.5 |
| Sepp1 | selenoprotein P, plasma, 1 | 1.0 | 1.4 |
| Serpina1 | serine (or cysteine) proteinase inhibitor, clade A, member 1 | 1.9 | 8.2 |
| Serpinb6 | serine (or cysteine) proteinase inhibitor, clade B (ovalbumin), member 6 | 0.9 | 0.7 |
| Serpine1 | serine (or cysteine) proteinase inhibitor, member 1 | 0.7 | 1.2 |
| Serpine1 | serine (or cysteine) proteinase inhibitor, member 1 | 0.3 | 1.0 |
| Serpinf1 | serine (or cysteine) proteinase inhibitor, clade F), member 1 | 1.3 | 3.6 |
| Serpinf1 | serine (or cysteine) proteinase inhibitor, clade F), member 1 | 1.3 | 3.2 |
| Serping1 | serine (or cysteine) proteinase inhibitor, clade G (C1 inhibitor), member 1, (angioedema, hereditary) | 1.2 | 2.4 |
| Sfmbt | Scm-related gene containing four mbt domains | 0.8 | 0.7 |
| Sfrp4 | secreted frizzled-related protein 4 | 1.2 | 5.0 |
| Sgk | serum/glucocorticoid regulated kinase | 1.0 | 0.8 |
| Sgne1 | secretory granule neuroendocrine protein 1 | 1.0 | 0.3 |
| Sh3bp5 | SH3-domain binding proteins 5 (BTK-associated) | 1.4 | 1.9 |
| Sh3d4 | SH3 domain protein 4 | 1.0 | 0.9 |
| Shank1 | EST211334 Normalized rat brain, Bento Soares Rattus sp. cDNA clone RBRBY29 3′ end, mRNA sequence. | 0.8 | 0.9 |
| Shc1 | SHC (Src homology 2 domain-containing) transforming protein 1 | 1.1 | 1.4 |
| Shox2 | short stature homeobox 2 | 0.8 | 0.7 |
| Siat10 | sialyltransferase 10 (alpha-2,3-sialyltransferase VI) | 1.0 | 0.6 |
| Siat7A | Similar to alpha-N-acetylgalactosaminide alpha-2,6-sialyltransferase (EC 2.4.99.3) I - mouse (LOC287920), mRNA | 0.7 | 1.0 |
| Siat9 | sialyltransferase 9 (CMP-NeuAc:lactosylceramide alpha-2,3-sialyltransferase; GM3 synthase) | 1.0 | 0.7 |
| Sip1 | survival of motor neuron protein interacting protein 1 | 0.9 | 0.8 |
| Sip30 | SNAP25 interacting protein 30 | 1.0 | 0.8 |
| Skap55 | Transcribed sequences | 1.3 | 0.7 |
| Slc10a2 | solute carrier family 10, member 2 | 1.0 | 0.4 |
| Slc11a2 | solute carrier family 11 member 2 | 1.2 | 1.4 |
| Slc12a2 | solute carrier family 12, member 2 | 1.1 | 1.5 |
| Slc16a10 | solute carrier family 16, member 10 | 1.0 | 0.6 |
| Slc1a3 | solute carrier family 1, member 3 | 1.1 | 0.7 |
| Slc20a1 | solute carrier family 20 (phosphate transporter), member 1 | 0.6 | 0.7 |
| Slc20a2 | solute carrier family 20, member 2 | 1.2 | 1.3 |
| Slc21a11 | solute carrier family 21 (organic anion transporter), member 11 | 1.1 | 1.8 |
| Slc21a11 | solute carrier family 21 (organic anion transporter), member 11 | 0.9 | 1.8 |
| Slc22a3 | solute carrier family 22, member 3 | 1.3 | 0.7 |
| Slc25a4 | solute carrier family 25 (mitochondrial adenine nucleotide translocator) member 4 | 1.0 | 0.8 |
| Slc25a5 | solute carrier family 25 (mitochondrial carrier; adenine nucleotide translocator), member 5 | 1.2 | 1.3 |
| Slc29a1 | solute carrier family 29, member 1 | 1.0 | 1.3 |
| Slc2a1 | solute carrier family 2, member 1 | 0.7 | 0.8 |
| Slc2a3 | solute carrier family 2, member 2 | 0.9 | 1.4 |
| Slc38a2 | solute carrier family 38, member 2 | 0.9 | 0.8 |
| Slc38a3 | solute carrier family 38, member 3 | 0.9 | 0.5 |
| Slc39a1 | solute carrier family 39 (iron-regulated transporter), member 1 | 1.5 | 2.7 |
| Slc39a1 | solute carrier family 39 (iron-regulated transporter), member 1 | 1.4 | 2.4 |
| Slc39a1 | solute carrier family 39 (iron-regulated transporter), member 1 | 1.4 | 1.8 |
| Slc3a2 | solute carrier family 3, member 2 | 0.9 | 1.2 |
| Slc5a3 | solute carrier family 5 (inositol transporters), member 3 | 0.7 | 0.4 |
| Slc6a6 | solute carrier family 6, member 6 | 0.5 | 0.5 |
| Slc7a1 | solute carrier family 7, member 1 | 0.8 | 1.0 |
| Slc7a1 | solute carrier family 7, member 1 | 0.7 | 0.9 |
| Slc8a3 | solute carrier family 8 (sodium/calcium exchanger), member 3 | 0.8 | 0.7 |
| Slpa | septin-like protein | 1.0 | 1.2 |
| Smhs2 | Smhs2 protein | 0.8 | 0.8 |
| Smoh | smoothened homolog (Drosophila) | 0.9 | 0.6 |
| Smoh | smoothened homolog (Drosophila) | 0.9 | 0.5 |
| Smyd2 | SET and MYND domain containing 2 | 1.0 | 0.8 |
| Snk | serum-inducible kinase | 1.1 | 1.8 |
| Snrk | SNF related kinase | 0.9 | 0.9 |
| Soat1 | acyl-coenzyme A:cholesterol acyltransferase | 0.7 | 0.7 |
| Sod1 | superoxide dismutase 1 | 1.0 | 0.9 |
| Sod2 | superoxide dismutase 2 | 1.3 | 1.9 |
| Sod2 | DRNBUB09 Rat DRG Library Rattus norvegicus cDNA clone DRNBUB09 5′, mRNA sequence. | 1.1 | 1.6 |
| Sp1 | Sp1 transcription factor | 0.8 | 0.8 |
| Sp17 | sperm autoantigenic protein 17 | 1.1 | 0.7 |
| Spa1 | SPA-1 like protein p1294 | 0.9 | 0.9 |
| Sparc | secreted acidic cysteine rich glycoprotein | 0.9 | 0.8 |
| Sparcl1 | SPARC-like 1 | 1.2 | 1.6 |
| Spna2 | alpha-spectrin 2 | 1.1 | 1.1 |
| Spnr | double-stranded RNA-binding protein p74 | 0.9 | 0.7 |
| Spnr | double-stranded RNA-binding protein p74 | 0.8 | 0.6 |
| Sponf | f-spondin | 2.1 | 7.4 |
| Spp1 | secreted phosphoprotein 1 | 1.3 | 1.5 |
| Sqle | squalene epoxidase | 1.1 | 1.4 |
| Sqstm1 | sequestosome 1 | 0.7 | 0.6 |
| Srpx | sushi-repeat-containing protein | 1.1 | 1.5 |
| Sstr4 | somatostatin receptor 4 | 0.8 | 0.7 |
| Ssx2ip | synovial sarcoma, X breakpoint 2 interacting protein | 1.1 | 1.3 |
| St14 | suppression of tumorigenicity 14 | 0.9 | 0.7 |
| Stat3 | signal transducer and activator of transcription 3 | 1.2 | 1.5 |
| Stat5b | signal transducer and activator of transcription 5B | 0.9 | 0.8 |
| Stg | small glutamine-rich tetratricopeptide repeat (TPR) containing protein (SGT) | 1.1 | 1.2 |
| Stk10 | serine/threonine kinase 10 | 1.1 | 1.3 |
| Stk17b | serine/threonine kinase 17b (apoptosis-inducing) | 1.4 | 2.4 |
| Stk17b | serine/threonine kinase 17b (apoptosis-inducing) | 1.4 | 2.3 |
| Stk17b | serine/threonine kinase 17b (apoptosis-inducing) | 1.3 | 2.3 |
| Stk2 | serine/threonine kinase 2 | 1.0 | 1.1 |
| Stmn1 | stathmin 1 | 1.8 | 2.0 |
| Stmn4 | stathmin-like 4 | 1.3 | 1.6 |
| Strn | striatin | 0.9 | 0.8 |
| Stx6 | syntaxin 6 | 1.0 | 1.3 |
| Stxbp2 | syntaxin binding protein Munc18-2 | 1.0 | 0.8 |
| Suox | sulfite oxidase | 0.7 | 0.8 |
| Surf1 | Surfeit 1 | 0.9 | 0.8 |
| Syt11 | synaptotagmin 11 | 0.9 | 0.9 |
| Taa1 | tumor-associated antigen 1 | 0.8 | 2.1 |
| Tagln | transgelin | 1.8 | 3.7 |
| Tc10 | ras-like protein | 1.3 | 1.5 |
| Tc10 | ras-like protein | 1.3 | 1.4 |
| Tcea2 | transcription elongation factor A2 | 0.8 | 0.8 |
| Tcf8 | transcription factor 8 | 1.0 | 1.2 |
| Tcf8 | transcription factor 8 | 1.0 | 1.1 |
| Tcirg1 | T-cell, immune regulator 1, ATPase, H+ transporting, lysosomal V0 protein a isoform 3 | 1.5 | 1.6 |
| Tcn2 | transcobalamin II precursor | 1.6 | 3.9 |
| Tdg | thymine-DNA glycosylase | 1.0 | 1.1 |
| Tesk2 | testis-specific kinase 2 | 0.8 | 0.7 |
| Tfb1m | Similar to sif and Tiam1-like exchange factor (LOC308142), mRNA | 1.2 | 1.0 |
| Tff3 | trefoil factor 3 | 0.9 | 0.8 |
| Tfpi2 | tissue factor pathway inhibitor 2 | 1.3 | 4.5 |
| Tfrc | transferrin receptor | 2.3 | 1.7 |
| Tfrc | transferrin receptor | 1.8 | 1.4 |
| Tgfa | transforming growth factor alpha | 2.6 | 3.6 |
| Tgfa | transforming growth factor alpha | 2.2 | 3.1 |
| Tgfb1i4 | transforming growth factor beta 1 induced transcript 4 | 1.3 | 1.4 |
| Tgfb2 | transforming growth factor, beta 2 | 0.9 | 1.7 |
| Tgfb2 | transforming growth factor, beta 2 | 0.8 | 1.3 |
| Tgfb3 | transforming growth factor, beta 3 | 0.7 | 0.7 |
| Tgm2 | tissue-type transglutaminase | 1.5 | 2.4 |
| Tgm2 | tissue-type transglutaminase | 1.0 | 1.6 |
| Thbd | thrombomodulin | 1.2 | 3.3 |
| Thbs4 | thrombospondin 4 | 1.0 | 2.3 |
| Thy1 | thymus cell antigen 1, theta | 0.8 | 1.9 |
| Tieg | TGFB inducible early growth response | 1.1 | 1.8 |
| Timm22 | translocase of inner mitochondrial membrane 22 homolog (yeast) | 1.2 | 1.3 |
| Timm23 | translocase of inner mitochondrial membrane 23 homolog (yeast) | 1.2 | 1.4 |
| Timp1 | tissue inhibitor of metalloproteinase 1 | 1.2 | 2.3 |
| Timp2 | tissue inhibitor of metalloproteinase 2 | 0.9 | 1.2 |
| Tjp1 | tight junction protein 1 | 1.0 | 1.4 |
| Tlr2 | toll-like receptor 2 variant 1 | 1.2 | 2.4 |
| Tmod2 | tropomodulin 2 | 0.9 | 0.6 |
| Tnfip6 | tumor necrosis factor induced protein 6 | 1.2 | 4.0 |
| Tnfrsf11b | tumor necrosis factor receptor superfamily, member 11b (osteoprotegerin) | 1.1 | 0.7 |
| Tnfrsf12a | tumor necrosis factor receptor superfamily, member 12a | 1.9 | 4.6 |
| Tnfrsf1a | tumor necrosis factor receptor superfamily, member 1a | 0.8 | 1.3 |
| Tnfsf11 | tumor necrosis factor (ligand) superfamily, member 11 | 1.2 | 2.9 |
| TOM70 | Similar to mKIAA0719 protein (LOC304017), mRNA | 1.3 | 1.3 |
| Top2a | topoisomerase (DNA) 2 alpha | 1.8 | 2.0 |
| Top2a | topoisomerase (DNA) 2 alpha | 1.6 | 1.9 |
| Top2a | topoisomerase (DNA) 2 alpha | 1.0 | 0.8 |
| Tpm1 | tropomyosin 1, alpha | 1.5 | 1.4 |
| Tpm1 | tropomyosin 1, alpha | 1.4 | 1.4 |
| Tpm1 | tropomyosin 1, alpha | 1.2 | 0.1 |
| Tpm1 | tropomyosin 1, alpha | 1.1 | 0.1 |
| Tpm3 | Rattus norvegicus tropomyosin 3, gamma (Tpm3), mRNA. | 0.4 | 0.3 |
| Tpo1 | developmentally regulated protein TPO1 | 1.3 | 1.6 |
| Trhr | thyrotropin releasing hormone receptor | 0.7 | 0.3 |
| Trpc2 | transient receptor potential cation channel, subfamily C, member 2 | 0.9 | 0.8 |
| Trrp6 | Rattus norvegicus trp6C gene for transient receptor potential Ca2+ channel 6C, complete cds. | 1.2 | 2.6 |
| TSP-2 | thrombospondin 2 | 0.9 | 1.4 |
| Tspan2 | tetraspan 2 | 1.4 | 2.3 |
| Tspan2 | tetraspan 2 | 1.2 | 1.7 |
| Tst | thiosulfate sulfurtransferase | 0.6 | 0.3 |
| Tsx | testis specific X-linked gene | 1.5 | 0.4 |
| Tuba1 | Similar to tubulin, alpha 6; tubulin alpha 6 (LOC300218), mRNA | 1.3 | 1.5 |
| Txn2 | thioredoxin 2 | 1.0 | 0.9 |
| Tyms | thymidylate synthase | 1.5 | 1.4 |
| Ua20 | putative UA20 protein | 1.1 | 1.6 |
| Uba52 | ubiquitin A-52 residue ribosomal protein fusion product 1 | 1.1 | 0.9 |
| Ubc | polyubiquitin | 1.0 | 0.9 |
| Ubd | ubiquitin D | 1.0 | 0.9 |
| Ube2n | ubiquitin-conjugating enzyme E2N (homologous to yeast UBC13) | 1.1 | 1.3 |
| Ube2v2 | ubiquitin-conjugating enzyme E2 variant 2 | 1.0 | 0.8 |
| Ube4a | ubiquitin conjugation factor E4 A | 0.9 | 0.7 |
| Ubqln1 | ubiquilin 1 | 1.0 | 1.2 |
| Ugdh | UDP-glucose dehydrogeanse | 1.0 | 1.3 |
| Uncl19 | UNC-119 homolog (C. elegans) | 0.9 | 0.7 |
| Uox | urate oxidase | 1.0 | 1.0 |
| Ush1c | Similar to harmonin isoform b3 (LOC308596), mRNA | 0.7 | 0.4 |
| Vamp1 | vesicle-associated membrane protein 1 | 1.1 | 1.7 |
| Vamp5 | vesicle-associated membrane protein 5 | 0.9 | 0.6 |
| Vamp8 | vesicle-associated membrane protein 8 (endobrevin) | 1.0 | 0.8 |
| Vdup1 | upregulated by 1,25-dihydroxyvitamin D-3 | 1.0 | 0.8 |
| Vkorc1 | vitamin K epoxide reductase complex subunit 1 | 1.0 | 0.9 |
| Vldlr | very low density lipoprotein receptor | 0.7 | 1.0 |
| Vldlr | very low density lipoprotein receptor | 0.7 | 0.9 |
| Vmp1 | vacuole Membrane Protein 1 | 1.2 | 1.7 |
| Vmp1 | vacuole Membrane Protein 1 | 1.1 | 1.7 |
| Waspip | Wiskott-Aldrich syndrome protein interacting protein | 0.8 | 1.1 |
| Wisp2 | WNT1 inducible signaling pathway protein 2 | 1.1 | 3.8 |
| Wrb | tryptophan rich basic protein | 0.9 | 0.7 |
| X2cr1 | pituitary tumor X2CR1 protein | 0.9 | 0.6 |
| X2cr1 | UI-R-AE1-zi-c-03-0-UI.s1 UI-R-AE1 Rattus norvegicus cDNA clone UI-R-AE1-zi-c-03-0-UI 3′, mRNA sequence. | 0.8 | 0.5 |
| Xpo1 | exportin 1 (CRM1, yeast, homolog) | 0.8 | 0.8 |
| Xrcc5 | X-ray repair complementing defective repair in Chinese hamster cells 5 | 0.8 | 0.9 |
| Yme1l1 | YME1 (S. cerevisiae)-like 1 | 1.2 | 1.2 |
| Ywhag | tyrosine 3-monooxgenase/tryptophan 5-monooxgenase activation protein, gamma polypeptide | 0.9 | 1.1 |
| Ywhah | tyrosine 3-monooxygenase/tryptophan 5-monooxygenase activation protein, eta polypeptide | 1.2 | 1.4 |
| Ywhaq | tyrosine 3-monooxygenase/tryptophan 5-monooxygenase activation protein, theta polypeptide | 1.1 | 1.4 |
| Zfp36l1 | zinc finger protein 36, C3H type-like 1 | 1.0 | 1.4 |
| Znf386 | zinc finger protein 386 (Kruppel-like) | 1.1 | 0.8 |

To gain perspective on classes of genes differentially regulated in ipsilateral OA cartilage (thereby potentially implicated in pathogenesis), gene ontology (GO) analysis was performed to assess the distribution of these genes across several functional groups. Of the annotated genes found, 52% were involved in metabolism, 25% in cell communication, 12% in cell differentiation, and 11% in transcription (FIG. 4C). More specific classifications were assessed, demonstrating differential regulation of genes encoding ECM (extra-cellular matrix) proteins, cytoskeletal components, receptors, ligands, growth factors, cytokines, cell cycle proteins, proteolytic enzymes and apoptosis factors (FIG. 4D). Interestingly, most of the dysregulated genes found in the categories of ECM molecules, ligands, cytokines, growth factors and proteloytic enzymes were up-rather than down-regulated, correlating with expected profiles in OA. As an example, Table 2 shows a detailed list of differentially regulated cytokines and growth factors in OA cartilage. The distribution of differentially regulated genes in OA cartilage shows which cellular processes are implicated at this early stage of disease in the model.

TABLE 2

| Name | Description | Gene Ontology: Molecular Function | Fold Change |
|---|---|---|---|
| Bmp3 | Bone morphogenetic protein 3 | Cytokine activity; Growth factor activity | 1.8 |
| Bmp4 | Bone morphogenetic protein 4 | Cytokine activity; Growth factor activity | 0.6 |
| Ccl2 | Chemokine (C-C motif) ligand 2 | G-protein-coupled receptor binding; Chemokine | 18.8 |
| | | activity; Cytokine activity; Protein binding | |
| Cklf1 | Chemokine-like factor 1 | Cytokine activity | 2.1 |
| Cklf1 | Chemokine-like factor 1 | Cytokine activity | 1.8 |
| Ddt | D-dopachrome tautomerase | Cytokine activity; Dopachrome isomerase activity | 0.6 |
| Dtr | Diphtheria toxin receptor | Growth factor activity; Heparin binding | 2.9 |
| Egf | Epidermal growth factor | Calcium ion binding; Epidermal growth factor receptor | 0.5 |
| | | binding; Growth factor activity | |
| Esm1 | Endothelial cell-specific molecule 1 | Insulin-like growth factor binding | 3.3 |
| F3 | Coagulation factor 3 | Hematopoietin/interferon-class (D200-domain) cytokine | 9.8 |
| | | receptor activity | |
| Hdgfrp3 | Hepatoma-derived growth factor, related | | 2.6 |
| | protein 3 | | |
| Igf1 | Insulin-like growth factor 1 | Growth factor activity; Hormone activity | 6.7 |
| Igf2 | Insulin-like growth factor 2 | Growth factor activity; Hormone activity | 0.6 |
| Igfbp3 | Insulin-like growth factor binding protein 3 | Growth factor binding: Insulin-like growth factor binding | 4.2 |
| Igfbp6 | Insulin-like growth factor binding protein 6 | | 2.9 |
| Igfbp6 | Insulin-like growth factor binding protein 6 | Growth factor binding; Insulin-like growth factor binding | 2.8 |
| Inhba | Inhibin beta-A | Growth factor activity; Hormone activity | 8.4 |
| Irs3 | Insulin receptor substrate 3 | Insulin receptor binding | 0.6 |
| Jag1 | Jagged 1 | Notch binding | 1.5 |
| Kitl | Kit ligand | | 5.3 |
| Ltbp1 | Latent transforming growth factor beta | Calcium ion binding; Growth factor binding | 1.9 |
| | binding protein 1 | | |
| Ltbp2 | Latent transforming growth factor beta | Calcium ion binding; Growth factor binding; | 2.1 |
| | binding protein 2 | Oxidoreductase activity | |
| Nudt6 | Antisense basic fibroblast growth factor | | 0.5 |
| Pdgfrb | Platelet derived growth factor receptor, | | 1.9 |
| | beta polypeptide | | |
| Spp1 | Secreted phosphoprotein 1 | Chemoattractant activity; Cytokine activity; Growth | 1.5 |
| | | factor activity; Integrin binding | |
| Tgfa | Transforming growth factor alpha | Growth factor activity | 3.6 |
| Tgfa | Transforming growth factor alpha | | 3.1 |
| Tgfb2 | Transforming growth factor, beta 2 | | 1.7 |
| Tnfsf11 | Tumor necrosis factor (ligand) | Cytokine activity; Receptor activity; Tumor necrosis | 2.9 |
| | superfamily, member 11 | factor receptor binding | |
| Wisp2 | WNT1 inducible signaling pathway protein 2 | Calcium ion binding; Insulin-like growth factor binding; | 3.8 |
| | | Phospholipase A2 activity; Protein binding | |

### Similarities to Chondrocyte Differentiation

Phenotypic alterations in articular chondrocytes at various stages of OA have been described in several studies (36, 37). These alterations are often reminiscent of chondrocyte differentiation in the growth plate. To assess the similarities of the osteoarthritic process with chondrocyte differentiation, the list of differentially expressed genes in OA cartilage (2-fold or greater) was compared to a list of differentially expressed genes (5-fold or greater) during in vitro chondrocyte differentiation in three-dimensional micromass culture (38) as shown in Table 3.

TABLE 3

| | Fold Change | | |
|---|---|---|---|
| | Ipsilateral | Chondrocyte | |
| Name | Cartilage | Differentiation | Description |
| Acta1 | 0.1 | 0.0 | Actin, alpha 1, skeletal muscle. |
| Bst1 | 2.7 | 12.3 | Bone marrow stromal cell |
| | | 7.5 | antigen 1 |
| C1s | 2.8 | 46.1 | Complement component 1, |
| | | | s subcomponent |
| Casq2 | 2.2 | 0.1 | Calsequestrin 2 |
| | 3.2 | | |
| Cd14 | 2.1 | 155.4 | CD14 antigen. |
| Cd36 | 2.3 | 15.7 | CD36 antigen |
| | | 14.4 | |
| Cd53 | 3.3 | 140.3 | CD53 antigen |
| Chi3l1 | 2.5 | 5.0 | Chitinase 3-like 1 |
| Chn2 | 0.4 | 0.1 | Chimerin (chimaerin) 2 |
| | 0.5 | | |
| Ctss | 2.7 | 30.1 | Cathepsin S |
| Cxcr4 | 2.8 | 5.8 | Chemokine (C—X—C motif) |
| | 3.3 | | receptor 4 |
| | 4.2 | | |
| Cybb | 2.3 | 41.3 | Cytochrome b-245, beta |
| | 2.8 | | polypeptide |
| Dbp | 0.1 | 11.7 | D site albumin promoter binding |
| | | | protein |
| Dcamkl1 | 2.3 | 8.0 | Double cortin and calcium/ |
| | | | calmodulin-dependent protein |
| | | | kinase-like 1 |
| Ecm1 | 2.0 | 6.6 | Extracellular matrix protein 1 |
| F3 | 9.8 | 5.7 | Coagulation factor III |
| Fcgr3 | 2.8 | 108.0 | Fc receptor, IgG, low affinity lib |
| | 3.0 | | |
| Gadd45a | 2.4 | 13.3 | Growth arrest and DNA- |
| | | | damage-inducible 45 alpha |
| Gap43 | 7.9 | 0.1 | Growth associated protein 43 |
| Gas6 | 0.5 | 8.0 | Growth arrest specific 6 |
| Gbp2 | 2.2 | 35.7 | Guanylate nucleotide binding |
| | | 15.5 | protein 2 |
| Gpm6b | 3.5 | 6.4 | Glycoprotein m6b |
| Hfe | 0.5 | 16.7 | Hemochromatosis |
| Igfbp6 | 2.8 | 28.7 | Insulin-like growth factor |
| | 2.9 | | binding protein 6 |
| Igsf6 | 2.3 | 13.4 | Immunoglobulin superfamily, |
| | | | member 6 |
| Il2rg | 4.0 | 7.7 | Interleukin 2 receptor, |
| | | 6.3 | gamma chain |
| Lbp | 6.1 | 23.4 | Lipopolysaccharide binding |
| | | | protein |
| Ltbp2 | 2.1 | 6.5 | Latent transforming growth |
| | | | factor beta binding protein 2 |
| Mcam | 2.4 | 0.1 | Melanoma cell adhesion |
| | | | molecule |
| Mg1 | 3.0 | 14.9 | Macrophage galactose N-acetyl- |
| | | | galactosamine specific lectin 1 |
| Mmp13 | 2.9 | 248.7 | Matrix metalloproteinase 13 |
| Mpeg1 | 2.2 | 51.8 | Macrophage expressed gene 1 |
| Mt1a | 2.2 | 9.1 | Metallothionein 1 |
| Nr1d1 | 0.3 | 6.0 | Nuclear receptor subfamily 1, |
| | | | group D, member 1 |
| Per3 | 0.3 | 9.4 | Period homolog 3 (Drosophila) |
| | | 5.3 | |
| Phex | 2.4 | 5.5 | Phosphate regulating gene with |
| | | | homologies to endopeptidases on |
| | | | the X chromosome |
| Ptprc | 2.2 | 107.3 | Protein tyrosine phosphatase, |
| | | | receptor type, C |
| Ptpro | 3.4 | 16.2 | Protein tyrosine phosphatase, |
| | | 8.0 | receptor type, O |
| Reln | 2.8 | 10.5 | Reelin |
| Rgs5 | 7.0 | 0.2 | Regulator of G-protein signaling |
| | | | 5 |
| Rgs5 | 10.0 | 0.2 | |
| Serping1 | 2.4 | 6.5 | Serine (or cysteine) proteinase |
| | | | inhibitor, clade G, member 1 |
| Tgm2 | 2.4 | 6.2 | Transglutaminase 2, C |
| | | 5.6 | polypeptide |
| | | 5.4 | |
| Thbd | 3.3 | 6.2 | Thrombomodulin |
| Thbs4 | 2.3 | 37.9 | Thrombospondin 4 |
| Tlr2 | 2.4 | 18.4 | Toll-like receptor 2 |
| Wisp2 | 3.8 | 34.4 | WNT1 inducible signaling |
| | | | pathway protein 2 |

Of the 46 genes common to both lists, 37 (80%) changed in the same direction during chondrocyte differentiation and in ipsilateral cartilage (2 genes down-regulated in both arrays, 35 genes up-regulated in both). Only 9 genes displayed opposing trends in the two settings. These data suggest that the cellular processes occurring during early OA are related to events of chondrocyte differentiation during development.

Genes up-regulated in both scenarios include the proteases Ctss, Reln and Mmp13 as well as other genes previously implicated in chondrocyte differentiation including Tgm2 (39), Ecm1 (40), Fcgr3 (41), Ltbp2 (42), and Phex (43). Another intriguing group up-regulated both during chondrocyte differentiation and in OA included genes involved in inflammation and chemokine/cytokine signaling, such as the LPS receptor Cd14, Cxcr4 and Il2rg and Tlr2. It was further demonstrated that CXCR4 is increased in ipsilateral OA chondrocytes as well as hypertrophic growth plate chondrocytes (FIG. 4d,e). Previous analyses (38) had also demonstrated that several members of the insulin-like growth factor (IGF) axis were dynamically regulated during chondrocyte differentiation, in agreement with their established roles in chondrocyte proliferation and hypertrophy (44, 45). The results show that Igf1, Igfbp3, and Igfbp6 were markedly up-regulated in ipsilateral cartilage (Tables 1 and 2).

### Discussion

In this study microarray technology and a recently developed rat model of OA (19) were used to determine differential gene expression indicative of OA pathogenesis.

The first histological signs of OA can be detected 4 weeks post-surgery. Cartilage degradation subsequently proceeds further, ultimately resulting in complete loss of articular cartilage. In the present experiments, gene expression analyses were performed at the 4-week time point to identify genes not previously known to be involved in OA pathogenesis. In fact, the present data represents the first genome-wide expression profile for early osteoarthritis in a mammalian model.

An important finding of the present study is that ACL-T/PM results in changes in gene expression in the contralateral joint. The majority of these changes were similar, though milder, to a subset of those observed in ipsilateral cartilage. This could be due to altered biomechanics (e.g. increased loading of the contralateral joint) or to systemic factors (e.g. inflammatory mediators) released in response to OA progression in the ipsilateral joint. Unsupervised clustering demonstrated greater heterogeneity in contralateral gene expression when compared to either sham controls or ipsilateral cartilage, with some animals showing little effects, while others showed significant changes. The reasons for this variability are unknown. More importantly, it was concluded that contralateral joints are not suitable controls in these studies.

Analyses of array data as well as confirmatory real-time PCR and immunofluorescence for known mediators of OA determined that the present model recapitulates established molecular events of human OA. This provides strong evidence that newly identified genes are also involved in OA progression. GeneOntology analyses demonstrated that many genes involved in cytokine and growth factor signaling are up-regulated in the operated joint. The dramatic change observed in the expression of chemokine (C—C motif) ligand 2 (Ccl2) (19-fold increase in ipsilateral cartilage) distinguished it as a diagnostic biomarker of OA. Additional chemokine signaling factors including chemokine-like factor 1 (Ckl1), chemokine (C—X3-C) ligand 1 (Cxc3) and chemokine (C—X—C motif) receptor 4 (Cxcr4) are also indicative of OA. Increased expression of Ednra, encoding endothelin receptor type A, is also shown to be a diagnostic biomarker of OA.

### REFERENCES

1. Berthiaume M J, Raynauld J P, Martel-Pelletier J, Labonte F, Beaudoin G, Bloch D A, et al. Meniscal tear and extrusion are strongly associated with progression of symptomatic knee osteoarthritis as assessed by quantitative magnetic resonance imaging. Ann Rheum Dis 2005; 64(4):556-563.
2. Englund M, Roos E M, Lohmander L S. Impact of type of meniscal tear on radiographic and symptomatic knee osteoarthritis: A sixteen-year followup of meniscectomy with matched controls. Arthritis & Rheumatism 2003; 48(8):2178-2187.
3. Gelber A C, Hochberg M C, Mead L A, Wang N Y, Wigley F M, Klag M J. Joint injury in young adults and risk for subsequent knee and hip osteoarthritis. Annals Of Internal Medicine 2000; 133(5):321-328.
4. Aigner T, Sachse A, Gebhard P M, Roach H I. Osteoarthritis: Pathobiology—targets and ways for therapeutic intervention. Advanced Drug Delivery Reviews; In Press, Corrected Proof.
5. Buckwalter J A, Martin J A. Osteoarthritis. Advanced Drug Delivery Reviews; In Press, Corrected Proof.
6. Lippiello L, Hall D, Mankin H J. Collagen synthesis in normal and osteoarthritic human cartilage. The Journal Of Clinical Investigation 1977; 59(4):593-600.
7. Eyre D R, McDevitt C A, Billingham M E, Muir H. Biosynthesis of collagen and other matrix proteins by articular cartilage in experimental osteoarthrosis. The Biochemical Journal 1980; 188(3):823-837.
8. Dessau W, Vertel B M, von der Mark H, von der Mark K. Extracellular matrix formation by chondrocytes in monolayer culture. J Cell Biol 1981; 90(1):78-83.
9. Wang J, Elewaut D, Hoffman I, Veys E M, Verbruggen G. Physiological levels of hydrocortisone maintain an optimal chondrocyte extracellular matrix metabolism. Ann Rheum Dis 2004; 63(1):61-66.
10. Hughes C E, Little C B, Buttner F H, Bartnik E, Caterson B. Differential Expression of Aggrecanase and Matrix Metalloproteinase Activity in Chondrocytes Isolated from Bovine and Porcine Articular Cartilage. J. Biol. Chem. 1998; 273(46):30576-30582.
11. Lohmander L S, Neame P J, Sandy JD. The structure of aggrecan fragments in human synovial fluid. Evidence that aggrecanase mediates cartilage degradation in inflammatory joint disease, joint injury, and osteoarthritis. Arthritis And Rheumatism 1993; 36(9):1214-1222.
12. Tchetina E V, Antoniou J, Tanzer M, Zukor D J, Poole A R. Transforming Growth Factor-{beta}2 Suppresses Collagen Cleavage in Cultured Human Osteoarthritic Cartilage, Reduces Expression of Genes Associated with Chondrocyte Hypertrophy and Degradation, and Increases Prostaglandin E2 Production. Am J Pathol 2006; 168(1):131-140.
13. Shuler F D, Georgescu H I, Niyibizi C, Studer R K, Mi Z, Johnstone B, et al. Increased matrix synthesis following adenoviral transfer of a transforming growth factor betal gene into articular chondrocytes. Journal Of Orthopaedic Research: Official Publication Of The Orthopaedic Research Society 2000; 18(4):585-592.
14. Fernandes J C, Martel-Pelletier J, Pelletier J-P. The role of cytokines in osteoarthritis pathophysiology. Biorheology 2002; 39(1-2):237-246.
15. Aida Y, Maeno M, Suzuki N, Shiratsuchi H, Motohashi M, Matsumura H. The effect of IL-1[beta] on the expression of matrix metalloproteinases and tissue inhibitors of matrix metalloproteinases in human chondrocytes. Life Sciences 2005; 77(25):3210-3221.
16. Lorenz H, Richter W. Osteoarthritis: Cellular and molecular changes in degenerating cartilage. Prog Histochem Cytochem 2006; 40(3):135-163.
17. Brandt K D. Animal models of osteoarthritis. Biorheology 2002; 39(1-2):221-35.
18. van den Berg W B. Lessons from animal models of osteoarthritis. Curr Opin Rheumatol 2001; 13(5):452-6.
19. Henry J L. Molecular events of chronic pain: from neuron to whole animal in an animal model of osteoarthritis. Novartis Found Symp 2004; 260:139-145.
20. Al-Shahrour F, Minguez P, Vaquerizas J M, Conde L, Dopazo J. BABELOMICS: a suite of web tools for functional annotation and analysis of groups of genes in high-throughput experiments. Nucl. Acids Res. 2005; 33(suppl-2):W460-464.
21. Vaquerizas J M, Conde L, Yankilevich P, Cabezon A, Minguez P, Diaz-Uriarte R, et al. GEPAS, an experiment-oriented pipeline for the analysis of microarray gene expression data. Nucl. Acids Res. 2005; 33(suppl2):W616-620.
22. Stanton L A, Sabari S, Sampaio A V, Underhill T M, Beier F. p38 MAP kinase signalling is required for hypertrophic chondrocyte differentiation. Biochem J 2004; 378(Pt 1):53-62.
23. Glasson S S, Askew R, Sheppard B, Carito B, Blanchet T, Ma H-L, et al. Deletion of active ADAMTS5 prevents cartilage degradation in a murine model of osteoarthritis. Nature 2005; 434(7033):644-648.
24. Malfait A-M, Liu R-Q, Ijiri K, Komiya S, Tortorella M D. Inhibition of ADAM-TS4 and ADAM-TS5 prevents aggrecan degradation in osteoarthritic cartilage. The Journal Of Biological Chemistry 2002; 277(25):22201-22208.
25. Duerr S, Stremme S, Soeder S, Bau B, Aigner T. MMP-2/gelatinase A is a gene product of human adult articular chondrocytes and is increased in osteoarthritic cartilage. Clin Exp Rheumatol 2004; 22(5):603-608.
26. Billinghurst R C, Dahlberg L, Ionescu M, Reiner A, Bourne R, Rorabeck C, et al. Enhanced cleavage of type II collagen by collagenases in osteoarthritic articular cartilage. The Journal Of Clinical Investigation 1997; 99(7):1534-1545.
27. Bau B, Gebhard P M, Haag J, Knorr T, Bartnik E, Aigner T. Relative messenger RNA expression profiling of collagenases and aggrecanases in human articular chondrocytes in vivo and in vitro. Arthritis And Rheumatism 2002; 46(10):2648-2657.
28. Mitchell P G, Magna H A, Reeves L M, Lopresti-Morrow L L, Yocum S A, Rosner P J, et al. Cloning, expression, and type II collagenolytic activity of matrix metalloproteinase-13 from human osteoarthritic cartilage. The Journal Of Clinical Investigation 1996; 97(3):761-768.
29. Recklies A D, Ling H, White C, Bernier S M. Inflammatory Cytokines Induce Production of CHI3L1 by Articular Chondrocytes. J. Biol. Chem. 2005; 280(50):41213-41221.
30. Mathy-Hartert M, Deby-Dupont G P, Reginster J Y L, Ayache N, Pujol J P, Henrotin Y E. Regulation by reactive oxygen species of interleukin-1 [beta], nitric oxide and prostaglandin E2 production by human chondrocytes. Osteoarthritis and Cartilage 2002; 10(7):547-555.
31. Abramson S B. The role of COX-2 produced by cartilage in arthritis. Osteoarthritis and Cartilage 1999; 7(4):380-381.
32. Loeser R F, Pacione C A, Chubinskaya S. The combination of insulin-like growth factor I and osteogenic protein I promotes increased survival of and matrix synthesis by normal and osteoarthritic human articular chondrocytes. Arthritis & Rheumatism 2003; 48(8):2188-2196.
33. Mankin H J, Johnson M E, Lippiello L. Biochemical and metabolic abnormalities in articular cartilage from osteoarthritic human hips. III. Distribution and metabolism of amino sugar-containing macromolecules. The Journal Of Bone And Joint Surgery. American Volume 1981; 63(1):131-139.
34. Benya P D, Padilla S R, Nimni M E. The progeny of rabbit articular chondrocytes synthesize collagen types I and III and type I trimer, but not type II. Verifications by cyanogen bromide peptide analysis. Biochemistry 1977; 16(5):865-872.
35. von der Mark K, Gauss V, von der Mark H, Muller P. Relationship between cell shape and type of collagen synthesised as chondrocytes lose their cartilage phenotype in culture. Nature 1977; 267(5611):531-532.
36. Fukui N, Purple C, Sandell L. Cell biology of osteoarthritis: the chondrocyte's response to injury. Curr Rheumatol Rep 2001; 3(6):496-505.
37. Drissi H, Zuscik M, Rosier R, 0'Keefe R. Transcriptional regulation of chondrocyte maturation: potential involvement of transcription factors in OA pathogenesis. Mol Aspects Med 2005; 26(3): 169-79.
38. James C G, Appleton C T G, Ulici V, Underhill T M, Beier F. Microarray Analyses of Gene Expression during Chondrocyte Differentiation Identifies Novel Regulators of Hypertrophy. Mol. Biol. Cell 2005; 16(11):5316-5333.
39. Johnson K A, Terkeltaub R A. External GTP-bound Transglutaminase 2 Is a Molecular Switch for Chondrocyte Hypertrophic Differentiation and Calcification. J. Biol. Chem. 2005; 280(15):15004-15012.

40. Deckers M M L, Smits P, Karperien M, Ni J, Tylzanowski P, Feng P, et al. Recombinant human extracellular matrix protein I inhibits alkaline phosphatase activity and mineralization of mouse embryonic metatarsals in vitro. Bone 2001; 28(1):14-20.
41. Nabbe K C A M, Boross P, Holthuysen A E M, Sloetjes A W, Kolls J K, Verbeek S, et al. Joint inflammation and chondrocyte death become independent of Fc? receptor type III by local overexpression of interferon-? during immune complex-mediated arthritis. Arthritis & Rheumatism 2005; 52(3):967-974.
42. Goessler U R, Bugert P, Bieback K, Deml M, Sadick H, Hormann K, et al. In-vitro analysis of the expression of TGFbeta-superfamily-members during chondrogenic differentiation of mesenchymal stem cells and chondrocytes during dedifferentiation in cell culture. Cell Mol Biol Lett 2005; 10(2):345-362.
43. Sabbagh Y, Carpenter T O, Demay M B. Hypophosphatemia leads to rickets by impairing caspase-mediated apoptosis of hypertrophic chondrocytes. PNAS 2005; 102(27):9637-9642.
44. Fisher M C, Meyer C, Garber G, Dealy C N. Role of IGFBP2, IGF-I and IGF-II in regulating long bone growth. Bone 2005; 37(6):741-750.
45. Tyler JA. Insulin-like growth factor 1 can decrease degradation and promote synthesis of proteoglycan in cartilage exposed to cytokines. The Biochemical Journal 1989; 260(2):543-548.
46. Hwang S-G, Yu S-S, Poo H, Chun J-S. c-Jun/Activator Protein-1 Mediates Interleukin-1 {beta}-induced Dedifferentiation but Not Cyclooxygenase-2 Expression in Articular Chondrocytes. J. Biol. Chem. 2005; 280(33):29780-29787.
47. Honorati M C, Cattini L, Facchini A. IL-17, IL-1[beta] and TNF-[alpha] stimulate VEGF production by dedifferentiated chondrocytes. Osteoarthritis and Cartilage 2004; 12(9):683-691.
48. Rothwell A G, Bentley G. Chondrocyte multiplication in osteoarthritic articular cartilage. J Bone Joint Surg Br 1973; 55-B(3):588-594.
49. Aigner T, Hemmel M, Neureiter D, Gebhard P M, Zeiler G, Kirchner T, et al. Apoptotic cell death is not a widespread phenomenon in normal aging and osteoarthritis human articular knee cartilage: a study of proliferation, programmed cell death (apoptosis), and viability of chondrocytes in normal and osteoarthritic human knee cartilage. Arthritis And Rheumatism 2001; 44(6):1304-1312.
50. Reichenberger E, Aigner T, von der Mark K, Sto H, Bertling W. In situ hybridization studies on the expression of type X collagen in fetal human cartilage. Developmental Biology 1991; 148(2):562-572.
51. Vornehm S I, Dudhia J, von der Mark K, Aigner T. Expression of collagen types 1× and XI and other major cartilage matrix components by human fetal chondrocytes in vivo. Matrix Biology 1996; 15(2):91-98.
52. Aigner T, Dietz U, Stoss H, von der Mark K. Differential expression of collagen types I, II, III, and X in human osteophytes. Laboratory Investigation; a Journal Of Technical Methods And Pathology 1995; 73(2):236-243.
53. Yuan G H, Masuko-Hongo K, Sakata M, Tsuruha J, Onuma H, Nakamura H, et al. The role of C—C chemokines and their receptors in osteoarthritis. Arthritis & Rheumatism 2001; 44(5): 1056-1070.
54. Villiger P M, Terkeltaub R, Lotz M. Monocyte chemoattractant protein-1 (MCP-1) expression in human articular cartilage. Induction by peptide regulatory factors and differential effects of dexamethasone and retinoic acid. J Clin Invest 1992; 90(2):488-496.
55. Mazzetti 1, Magagnoli G, Paoletti S, Uguccioni M, Olivotto E, Vitellozzi R, et al. A role for chemokines in the induction of chondrocyte phenotype modulation. Arthritis & Rheumatism 2004; 50(1):112-122.
56. Roy-Beaudry M, Martel-Pelletier J, Pelletier J-P, M'Barek K N, Fazool S C, Moldovan S F. Endothelin 1 promotes osteoarthritic cartilage degradation via matrix metalloprotease I and matrix metalloprotease 13 induction. Arthritis & Rheumatism 2003; 48(10):2855-2864.
57. Manacu C, Martel-Pelletier J, Roy-Beaudry M, Pelletier J-P, Fernandes J, Shipkolye F, et al. Endothelin-1 in osteoarthritic chondrocytes triggers nitric oxide production and upregulates collagenase production. Arthritis Res Ther. 2005; 7(2):R324-R332.
58. Nahir A M, Hoffman, A., Lorber, M., and Keiser, H. R. Presence of immunoreactive endothelin in synovial fluid: analysis of 22 cases. J Rheumatol 1991; 18(5):678-680.
59. Wharton J, Rutherford R A, Walsh D A, Mapp P I, Knock G A, Blake D R, et al. Autoradiographic localization and analysis of endothelin-1 binding sites in human synovial tissue. Arthritis & Rheumatism 1992; 35(8):894-899.
60. Khatib A M, Lomri A, Moldovan F, Soliman H, Fiet J, Mitrovic D R. Endothelin I receptors, signal transduction and effects on DNA and proteoglycan synthesis in rat articular chondrocytes. Cytokine 1998; 10(9):669-679.
61. Valdes A M, Hart D J, Jones K A, Surdulescu G, Swarbrick P, Doyle D V, et al. Association study of candidate genes for the prevalence and progression of knee osteoarthritis. Arthritis And Rheumatism 2004; 50(8):2497-2507.
62. Aigner T, Bartnik E, Zien A, Zimmer R. Functional genomics of osteoarthritis. Pharmacogenomics 2002; 3(5):635-650.

All references referred to herein are incorporated herein by reference.

We claim:

1. A method of diagnosing osteoarthritis in a mammal comprising the steps of:
i) obtaining a biological sample from the mammal; and
ii) quantifying in the sample the expression of at least one chondrocyte-specific gene or gene product, wherein a differential in expression of said gene or gene product in comparison with a standard is indicative of osteoarthritis.

2. A method as defined in claim 1, wherein the gene exhibits at least a 1.5-fold differential in expression.

3. A method as defined in claim 1, wherein the gene expression is upregulated.

4. A method as defined in claim 1, wherein the gene expression is down-regulated.

5. A method as defined in claim 1, wherein the chondrocyte-specific gene encodes an extra-cellular matrix protein.

6. A method as defined in claim 5, wherein the chondrocyte-specific gene encodes a cell-surface receptor.

7. A method as defined in claim 5, wherein the at least one chondrocyte-specific gene encodes a gene product selected from the group consisting of Cklf1, Inhba, Ccl2, Sfrp4, Ctsh, Ctss, Il2rg, Cd44, Pdgfrb, Fgfr2, Wisp2 and TGFα.

8. A method as defined in claim 1, wherein the expression of more than 1 chondrocyte-specific gene from the group consisting of Cklf1, Inhba, Ccl2, Sfrp4, Ctsh, Ctss, Il2rg, Cd44, Pdgfrb, Fgfr2, Wisp2, Ednra and TGFα is quantified.

9. A method as defined in claim 1, wherein the at least one chondrocyte-specific gene encodes a chemokine signaling factor.

10. A method as defined in claim 9, wherein the chemokine signaling factor is selected from the group consisting of Ckl1, Cxc3 and Cxcr4.

11. A kit for use in the diagnosis of osteoarthritis in a mammal, said kit comprising at least one probe directed to a chondrocyte-specific gene in the mammal that exhibits an increase in expression of at least about 1.5-fold.

12. A method of diagnosing osteoarthritis in a mammal comprising:
i) obtaining a biological sample from the mammal;
ii) quantifying in the sample the expression of multiple chondrocyte-specific genes or gene products to generate a chondrocyte-specific gene expression profile; and
iii) comparing the generated profile with a standard chondrocyte-specific gene expression profile generated from a biological sample obtained from a non-osteoarthritic mammal, wherein differential expression of one or more of said genes or gene products is indicative of osteoarthritis.

13. A method as defined in claim 12, wherein the differential expression is at least about 1.5 fold.

14. A method as defined in claim 12, wherein one or more of the chondrocyte-specific genes is upregulated.

15. A method as defined in claim 12, wherein one or more of the chondrocyte-specific genes encodes a chemokine signaling factor.

16. A method as defined in claim 12, wherein one or more of the chondrocyte-specific genes encodes an extra-cellular matrix protein.

17. A method as defined in claim 12, wherein one or more of the chondrocyte-specific genes encodes a cell-surface receptor.

18. A method as defined in claim 12, wherein the chondrocyte-specific gene products include one or more of a gene product selected from the group consisting of Cklf1, Inhba, Ccl2, Sfrp4, Ctsh, Ctss, Il2rg, Cd44, Pdgfrb, Fgfr2, Wisp2, Ednra, TGFα, Ckl1, Cxc3 and Cxcr4.


SEQUENCE LISTING (3 sequences)

SEQ ID NO: 1 - DNA, 19 bp, Artificial
  forward primer
gaaggtgaaggtcggagtc

SEQ ID NO: 2 - DNA, 20 bp, Artificial
  reverse primer
gaagatggtgatgggatttc

SEQ ID NO: 3 - DNA, 20 bp, Artificial
  Probe
caagcttcccgttctcagcc